(19) United States
(12) Patent Application Publication
(10) Pub. No.: US 2022/0298510 A1
(43) Pub. Date: Sep. 22, 2022

(54) COMPOUNDS AND METHODS FOR INHIBITING CANCERS VIA REST INHIBITION

(71) Applicant: Icahn School of Medicine at Mount Sinai, New York, NY (US)

(72) Inventors: Emily BERNSTEIN, New York, NY (US); Zulekha Qadeer, New York, NY (US); Dan Hasson, New York, NY (US)

(73) Assignee: ICAHN SCHOOL OF MEDICINE AT MOUNT SINAI, New York, NY (US)

(21) Appl. No.: 17/616,061

(22) PCT Filed: May 4, 2020
(86) PCT No.: PCT/US2020/031378

§ 371 Date: Dec. 2, 2021

Related U.S. Application Data
(60) Provisional application No. 62/856,489, filed on Jun. 3, 2019.

(51) Int. Cl. C12N 15/113; A61K 45/06; A61P 35/00; G01N 33/50; G01N 33/574

(52) U.S. Cl. CPC C12N 15/1135; A61K 45/06; A61P 35/00; G01N 33/5011; G01N 33/57407; G01N 2800/52

(57) ABSTRACT

Compositions and methods for diagnosing and treating cancers that have developed from neuronal cells and neuronal cell lines.

### RELATED APPLICATIONS

This application is a § 371 national stage of PCT International Application No. PCT/US20/31378, filed May 4, 2020, claiming the benefit of U.S. Provisional Patent Application Ser. No. 62/856,489, filed Jun. 3, 2019, which is hereby incorporated by reference in its entirety.

### TECHNICAL FIELD

The present invention relates generally to inhibition of wild-type and mutant forms of nucleotides and proteins involved in certain cancers, such as, neuroblastomas, gliomas, atypical teratoid rhabdoid tumors, and sarcomas. The present invention identifies REST as a therapeutic target that is activated in cancers and that promotes silencing of neuronal differentiation genes. The present invention also relates to therapeutic agents for the inhibition or treatment of cancer, methods of treatment, and diagnostic methods using the identified proteins and nucleotides.

### BACKGROUND

In the nucleus of eukaryotic cells, the vast majority of DNA is tightly packed to form chromatin during normal cellular function. Alteration of the chromatin structure can lead to transcription factor binding and gene transcription. Chromatin remodeling is highly regulated because uncontrolled remodeling can lead to gene dysregulation, resulting in senescence, apoptosis, changes in cellular fate and possibly cancer.

Neuroblastoma (NB) is an aggressive pediatric extracranial tumor thought to arise from impaired differentiation of progenitor cells of the peripheral nervous system. In adolescents and young adults, indolent or chronic NB is characterized by protracted disease progression and marked by the absence of MYCN amplifications. Alterations of ATRX (Alpha Thalassemia/Mental Retardation, X-linked), a SWI/SNF-like chromatin remodeling protein, are implicated in this disease.

The ATRX protein contains multiple highly conserved domains, including: an ADD (ATRX-DNMT3-DNMT3L) domain that binds trimethylated histone H3 at lysine 9 (H3K9me3) when unmethylated at H3K4; an HP1α-binding motif; a putative EZH2 interaction domain; a DAXX binding domain that mediates H3.3 deposition at heterochromatic regions (e.g. telomeres and repetitive DNA); and an ATPase domain, responsible for its remodeling activities. Large N-terminal deletions of ATRX can generate in-frame fusion (IFF) proteins, which are devoid of key chromatin interaction domains but retain the SWI/SNF-like helicase domain.

ATRX mutations are known to play a role in cancer; they are the most common recurring event in the indolent clinical subtype (˜30%) of neuroblastoma. ATRX mutations are also present in a variety of other tumor types, including for example, gliomas, pediatric glioblastoma multiforme, pediatric adrenocortical carcinoma, adrenocortical, and osteosarcoma. However, their exact role and their impact through epigenomic effects remain a mystery. ATRX mutations are associated with overall poor survival, and no effective therapies have been identified previously.

The current invention addresses these deficiencies. The inventors have demonstrated herein that ATRX IFF proteins have distinct genomic distribution compared to wild type (WT) ATRX protein and are absent from H3K9me3-enriched chromatin, yet they are bound to active promoters. The current invention shows a key role for REST (RE-1 Silencing Transcription Factor) as an ATRX IFF target that is activated and that promotes silencing of neuronal differentiation genes. Notably, the current invention also shows that ATRX IFF cells show exquisite sensitivity to EZH2 inhibitors (EZH2i) in vitro and in vivo, due in part to derepression of neurogenesis genes, including a subset of REST targets.

### SUMMARY OF THE TECHNOLOGY

The present invention identifies for the first time a transcription factor that: is correlated with certain cancers (such as neuroblastoma); is a target for inhibition; and responds to inhibition in both in vitro and in vivo models. In particular, the present invention identifies this transcription factor as guiding the suppressed neural differentiation state in NB. Inhibition of REST leads to apoptosis in ATRX IFF NB cells and restores neuronal differentiation programs in NB cells.

One aspect of the present invention provides a specific transcription factor that is involved in certain types of cancers, such as neuroblastomas, gliomas, and sarcomas.

Another aspect of the present invention provides therapeutic agents and compositions for the inhibition of cancer in a subject.

Another aspect of the invention provides therapeutic agents and compositions for the inhibition of the production or activity of REST in a cell.

Another aspect of the present invention provides a specific transcription factor that is a target for modulation to treat specific diseases, such as cancers.

Another aspect of the present invention provides a specific protein or nucleotide that is a target for modulation to treat certain diseases, such as cancers.

Another aspect of the present invention provides a biological marker for diagnosing diseases such as neuroblastoma, glioma, or sarcoma.

Another aspect of the present invention provides a method of screening for compositions and methods to treat or prevent certain diseases, such as cancers.

Another aspect of the present invention provides a therapeutic agent or composition for inhibiting proliferation or growth of neuronal cells, as well as a method for inhibiting such proliferation or accumulation.

The invention further provides a therapeutic method for treating diseases, such as cancer in a subject by inhibiting the activity or production of REST in a cell.

In one embodiment, the invention further provides a method for inhibiting REST production or activity using a small molecule, nucleotide or protein.

In one embodiment, the invention further provides a method for inhibiting REST production or activity using a vaccine or antibody.

The invention further provides a therapeutic method for treating disease in a subject by inhibiting the production or activity of EZH2 in a cell.

The invention further provides a therapeutic method for treating disease in a subject by inhibiting the production or activity of EZH2 in a neuronal cell.

The invention further provides a therapeutic method for treating cancer in a subject by inhibiting the production or activity of EZH2 in a cell.

The invention further provides a method for inhibiting EZH2 activity or production using a small molecule, nucleotide or protein.

The invention further provides a therapeutic agent that decreases REST (RE-1 Silencing Transcription Factor) production or activity in cancer cells.

The invention further provides a treatment method that combines decreasing REST production or activity with EZH2 inhibitors or degraders.

The invention further provides a therapeutic agent that comprises a nucleotide, chemical compound, protein, or combination thereof and a pharmaceutically acceptable carrier or diluent.

The invention further provides a therapeutic agent that acts as a REST degrader.

The invention further provides a REST degrader that is a nucleotide.

The invention further provides a REST degrader that is a shRNA, siRNA or LNA.

The invention further provides a therapeutic agent that decreases REST production or activity in ATRX mutant cancer cells.

The invention further provides a therapeutic agent wherein REST degradation occurs via a ubiquitin ligase complex and proteasome mediated degradation.

### BRIEF DESCRIPTION OF THE FIGURES

FIG. 1 shows that ATRX IFF proteins redistribute from H3K9me3-coupled regions to promoters of actively expressed genes, including REST.

FIG. 2 shows that ATRX IFF proteins in NB cells exhibit REST and H3K27me3 occupancy at repressed neuronal differentiation genes.

FIG. 3 shows that EZH2 inhibition in NB cells positive for ATRX IFF leads to cell death and tumor regression.

FIG. 4 shows that EZH2 inhibition in NB cells positive for ATRX IFF de-represses H3K27me3- and REST-bound neuronal differentiation genes.

FIG. 5 shows screening for NB cell lines positive for ATRX IFF proteins, PDX models, and tumor samples.

FIG. 6 shows analysis of ATRX IFF (CHLA-90 and SK-N-MM), LAN-6, and SK-N-FI cell lines for mutations, telomere status, and copy number variations.

FIG. 7 shows an integrated analysis of ChIP-seq and RNA-seq from NB cells and validation.

FIG. 8 shows ChIP-seq analysis of REST in NB cells, ChIP-seq analysis of H3K27me3 in NB cells, and characterization of ATRX IFF NB patient tumors.

FIG. 9 shows ChIP-seq analysis of H3K27me3 in NB cells and characterization of ATRX IFF NB patient tumors.

FIG. 10 shows that REST and EZH2 knockdown reduces proliferation NB cells positive for ATRX IFF.

FIG. 11 shows that EZH2i increases apoptosis in ATRX IFF NB cells.

FIG. 12 shows EZH2i cytotoxicity in NB cells in 2D and 3D cultures.

FIG. 13 shows RNA-seq analysis and validation of EZH2i-treated NB cells.

### DETAILED DESCRIPTION

Note that the term “subject” in the present invention is not particularly limited, and examples thereof include humans, mice, rats, cattle, horses, pigs, sheep, monkeys, dogs, and cats.

The term “composition” according to the present invention may be in the form of a vaccine, adjuvant, biological, pharmaceutical, pharmaceutical composition, a reagent used in an animal model, or a combination of such ingredients. The vaccine, adjuvant, biological, pharmaceutical, pharmaceutical composition, reagent, or combinatorial product can have the effect of inhibiting REST production or activity in a cell.

The term “LNA (locked nucleic acid)” according to the present invention are modified oligonucleotides that contain one or more nucleotide building blocks in which an extra methylene bridge “locks” the ribose moiety either in the C3′-endo or C2′-endo conformation.

The term “REST degrader” according to the present invention is a compound or molecule that binds to REST that accelerates the destruction or removal of REST protein from the cell via the ubiquitin-proteasome pathway.

The inventors have discovered the mechanisms whereby ATRX structural alterations contribute to indolent NB. In particular, the inventors found that ATRX IFFs generate truncated protein products retaining the SNF2 N helicase domain. The protein products no longer bind H3K9me3, and instead occupy active promoters including that of REST. They have, therefore, discovered how to identify, target, and treat these mutations in subjects.

The inventors further demonstrate that EZH2 inhibition induces upregulation of neuronal maturation genes, some of which are REST-bound, H3K27me3-enriched, and transcriptionally suppressed in ATRX IFF NB tumors and cell lines. They have also discovered that this culminates, at least in part, in selective apoptosis of ATRX IFF NB cells.

### EXAMPLES

Provided below are select examples of certain embodiments of the present invention; however, the invention is not limited to these examples or the specific embodiments recited above.

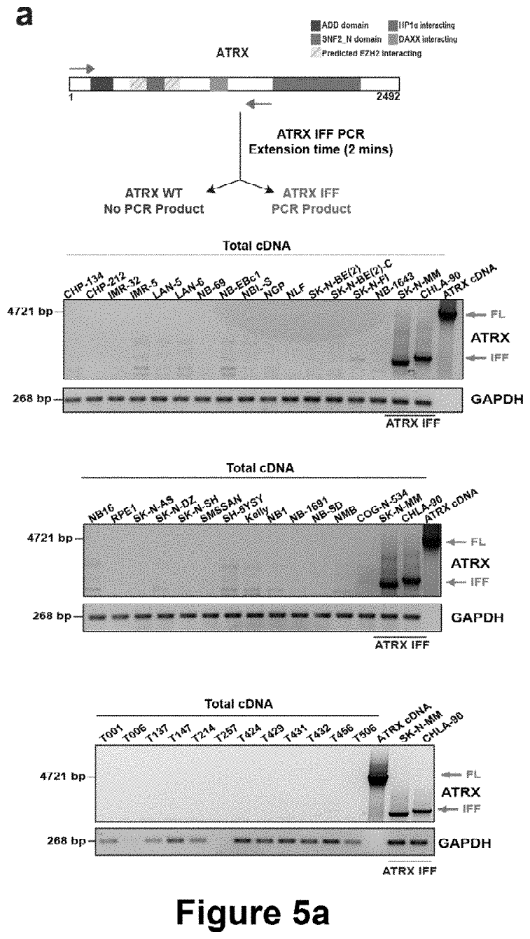

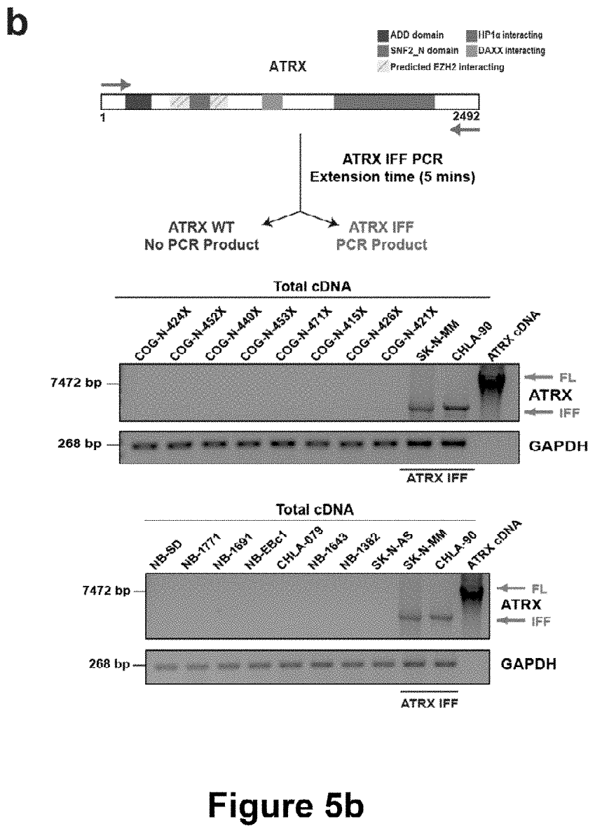

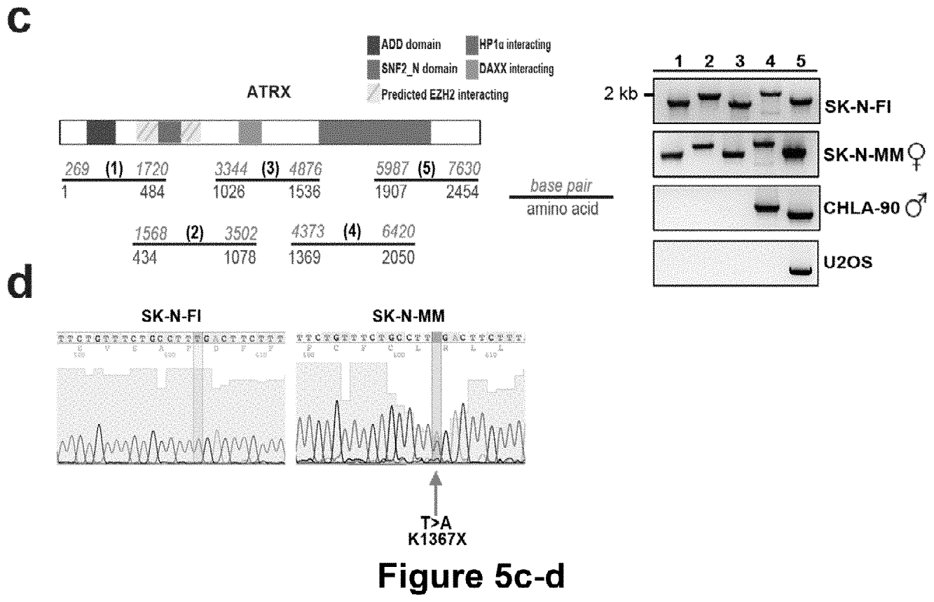

The inventors have discovered that the RE-1 Silencing Transcription Factor (REST) plays a critical role in certain types of tumors, such as neuroblastoma. The inventors screened an extensive panel of patient-derived cell lines, PDX models and human tumor samples to identify ATRX IFFs (FIG. 5).

FIG. 5 shows screening for NB cell lines positive for ATRX IFF proteins, PDX models, and tumor samples. (a), (b) Diagram illustrating screening strategy to identify ATRX IFFs. Schematic (top) with red arrows indicating primer locations on the ATRX gene product. Ethidium bromide-stained agarose gel (bottom panels) depicting ATRX PCR products amplified from total cDNA pool of NB cell lines, PDX models, and tumor samples. ATRX plasmid used as control for WT ATRX. GAPDH used to assess quality of cDNA. (c) Schematic (left) illustrating primer pairs and locations on ATRX gene product. Gel (right) depicting five amplicons for ATRX gene product. U2OS used as a positive control for deletions. (d) Fragment #3 of SK-N-MM was Sanger sequenced to identify K1367X mutation. Traces depicted on the right, SK-N-FI used as a WT control.

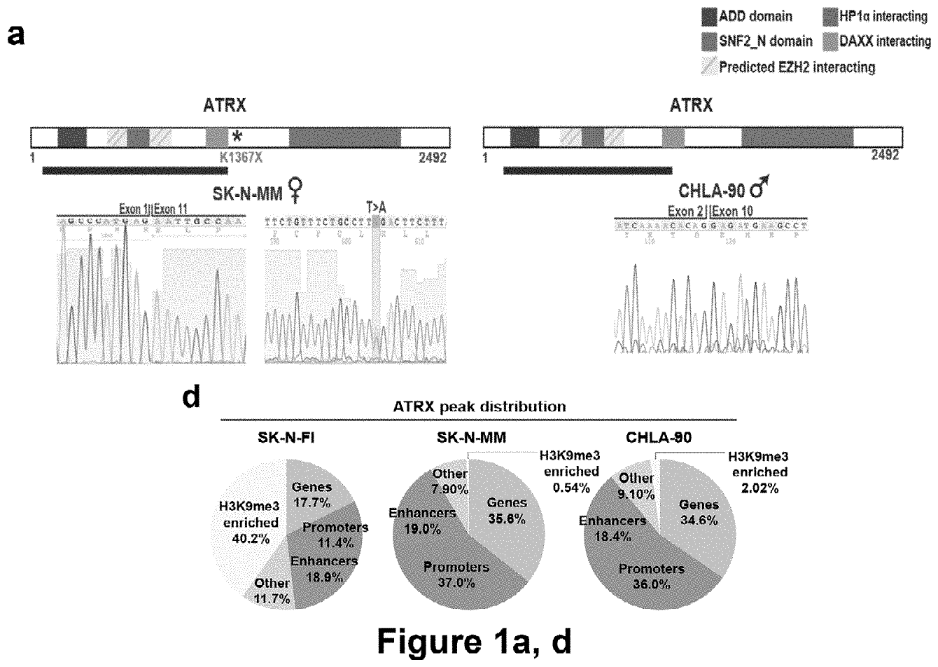

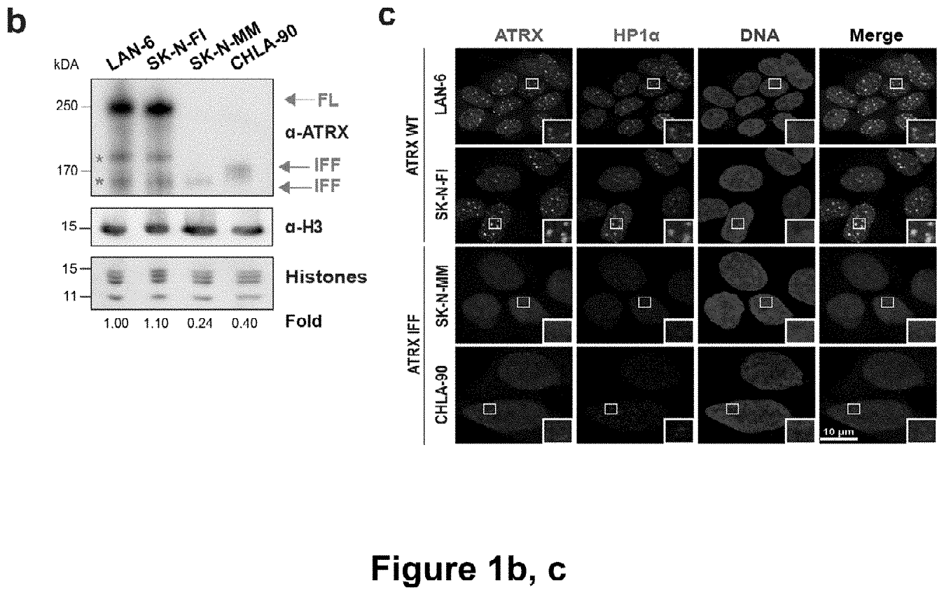

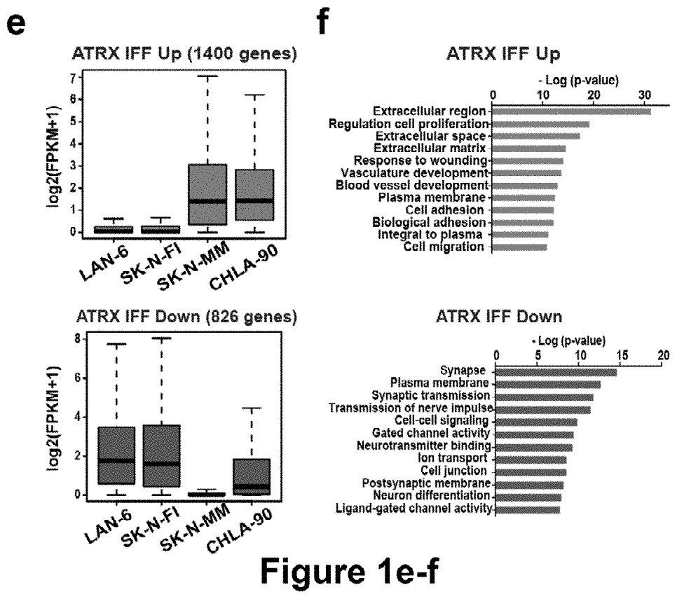

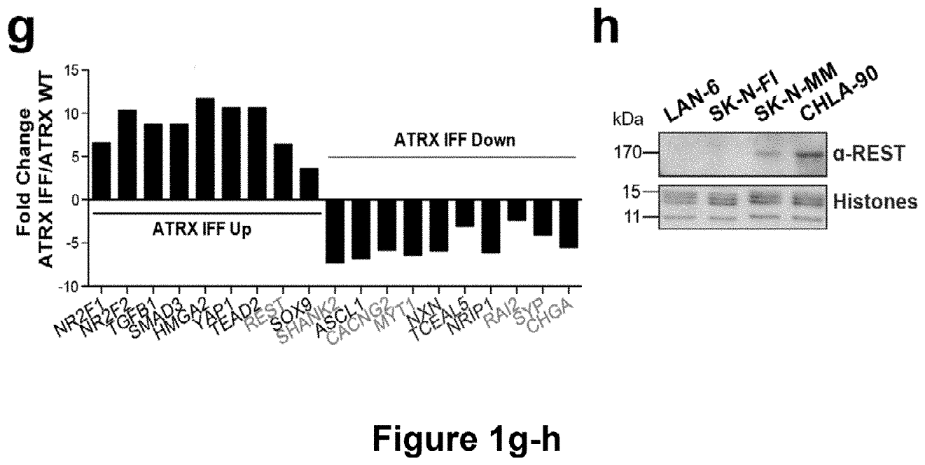

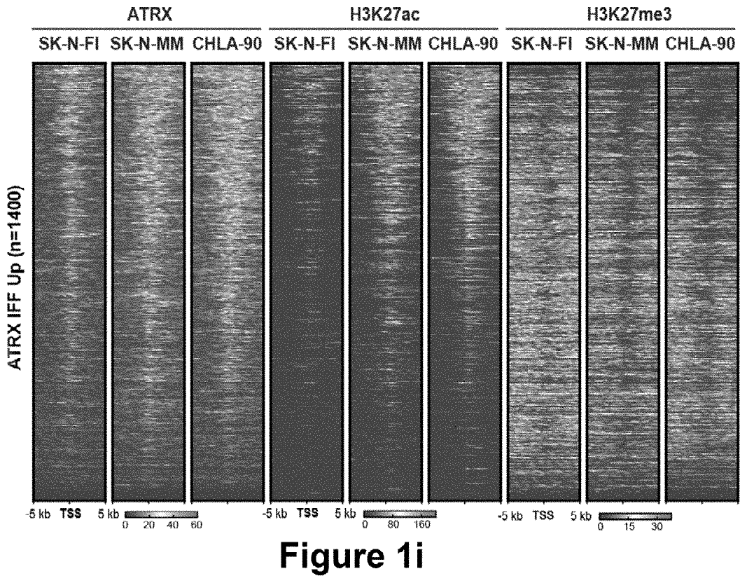

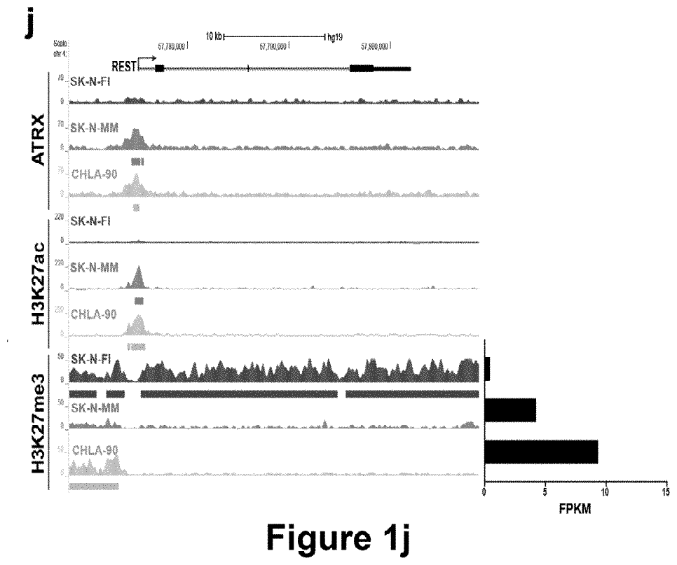

Utilizing PCR-based assays that favor amplification of an ATRX IFF gene product vs. full length ATRX from a total cDNA_pool, the inventors identified two human-derived NB cell lines, SK-N-MM and CHLA-90, which carry distinct structural variations in the ATRX gene (FIG. 1a, FIG. 5).

FIG. 1 shows that ATRX IFF proteins redistribute from H3K9me3-coupled regions to promoters of actively expressed genes. (a) Schematic of ATRX protein product with domains depicted and alterations identified in SK-N-MM and CHLA-90. Black bar underneath indicates N-terminal deletion. Sanger sequencing traces from SK-N-MM and CHLA-90 cDNA identifying ATRX alterations. (b) Immunoblot for ATRX demonstrating ATRX IFF protein products in the extracted chromatin fraction, indicated by red arrows. Asterisks denote breakdown products. H3 and histones used as a loading control. Protein fold-change measured relative to total H3. (c) Immunofluorescence for ATRX and HP1α in NB cells. Images taken at 40× with a 3× internal magnification, scale bar 10 μm. (d) Pie chart displaying the percentage of ATRX peaks occupying promoters, enhancers, gene bodies, H3K9me3 enriched and other regions for each cell line. Promoters: TSS±2 kb; gene bodies: TSS to TES; enhancers: called with levels of H3K27ac and overlapped with ATRX peaks; H3K9me3 enriched: ATRX peaks overlapped with H3K9me3 peaks; all other regions defined as other. (e) Box plots of expression levels of differential genes upregulated (n=1,400) and downregulated (n=826) in ATRX IFF NB cells. Statistical significance assessed using one-way ANOVA and summarized in Table 1. (f) Gene ontology (GO) analysis of ATRX IFF Up and ATRX IFF Down genes. Enriched terms ranked by most significant p-value. (g) Expression levels of representative genes upregulated in RNA-seq. Fold-change on log 2 scale of mean Fragments Per Kilobase Million (FPKM) of ATRX IFF cells over mean FPKM of ATRX WT cells plotted. REST target genes identified by ChIP-seq Enrichment Analysis (ChEA) labeled in red. (h) Immunoblots for REST in the extracted chromatin fraction of NB cells. Histones used as loading control. (i) Heatmap of ATRX, H3K27ac and H3K27me3 enrichment in the promoters of ATRX IFF Up genes, (promoters: TSS±2 kb). (j) UCSC Genome Browser snapshot of ATRX, H3K27ac, and H3K27me3 ChIP-seq at the REST locus (y axis=reads per kilobase per million reads). Significant peaks called by both MAC2 and annotated under each profile by colored bar (FDR=5×10⁻² for SKNFI, 5×10⁻³ for SKNMM, 5×10⁻⁴ for CHLA90 ATRX peaks; and 1×10⁻⁵ for all cell lines H3K27ac and H3K27me3 peaks).

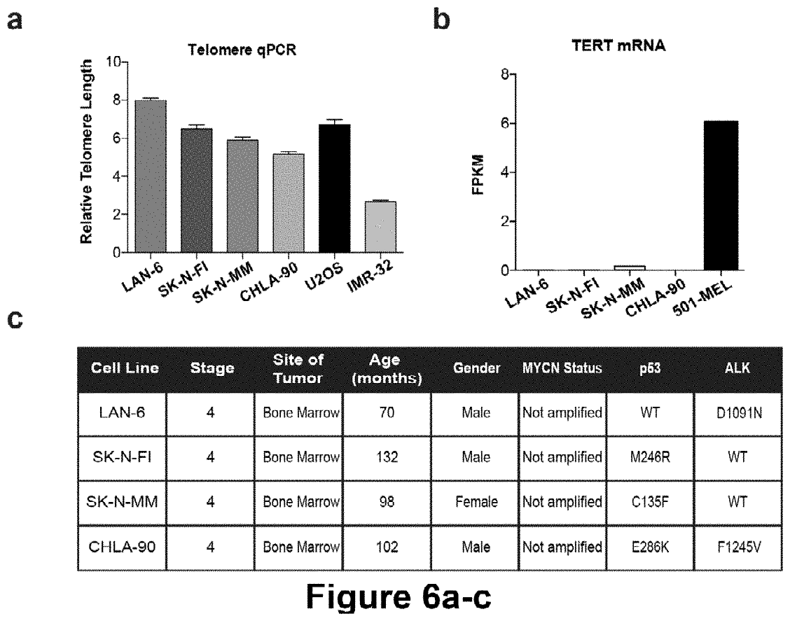

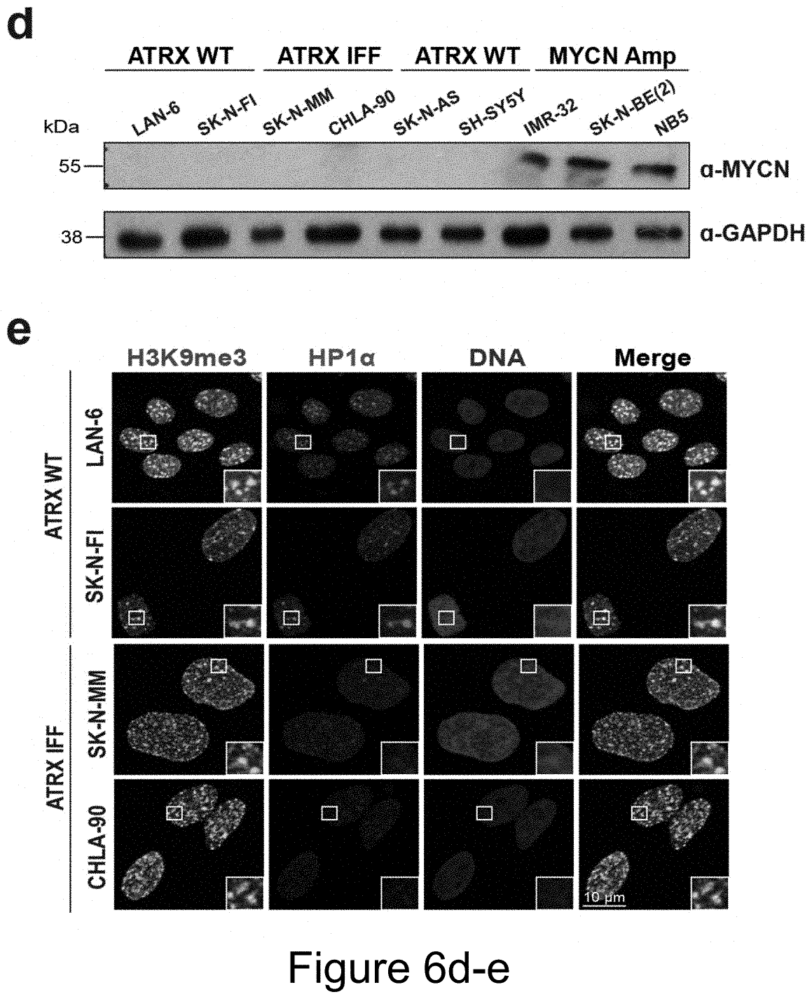

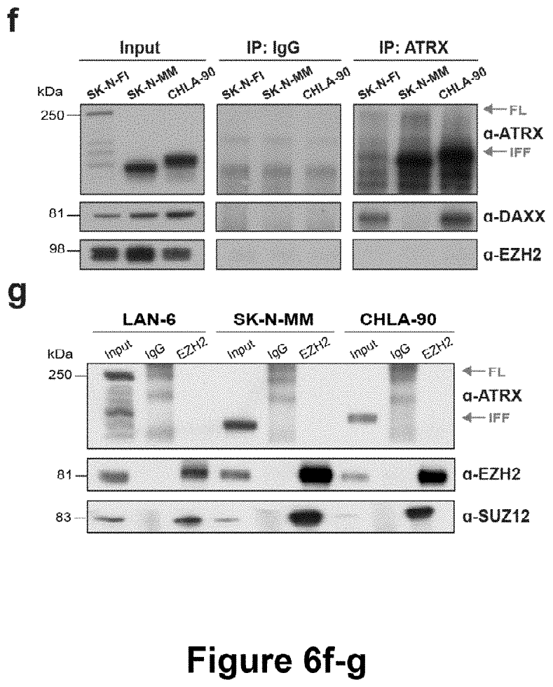

ATRX is located on the X chromosome, hence the male cell line CHLA-90 carries a single ATRX copy harboring an IFF (exon 2 to 10). The female cell line SK-N-MM harbors ATRX alterations on both alleles: an ATRX IFF (exon 1 to 11) and a nonsense mutation (K1367X) producing a truncated product predicted to be unstable (FIG. 1a, FIG. 5 c, d). The inventors characterized these two ATRX IFF cell lines derived from stage 4 NB, along with LAN-6 and SK-N-FI (ATRX WT; stage- and age-matched; MYCN non-amplified NB lines) for mutations and copy number variations, as well as telomere status. The inventors found that ATRX IFF NB cell lines display alternative lengthening of telomeres (ALT), are not amplified for MYCN, and harbor few recurrent mutations (FIG. 6a-d), similar to clinical observations.

Further testing shows that although ATRX IFFs lack key protein interaction domains (FIG. 1a), they remain chromatin bound, albeit at reduced levels compared to WT NB cell lines LAN-6 and SK-N-FI (FIG. 1b, c). The inventors found that while WT cell lines displayed ATRX localization in heterochromatin foci marked by HP1α and its cognate modification H3K9me3, ATRX IFF cells exhibited loss of ATRX- and HP1α-enriched foci (FIG. 1c).

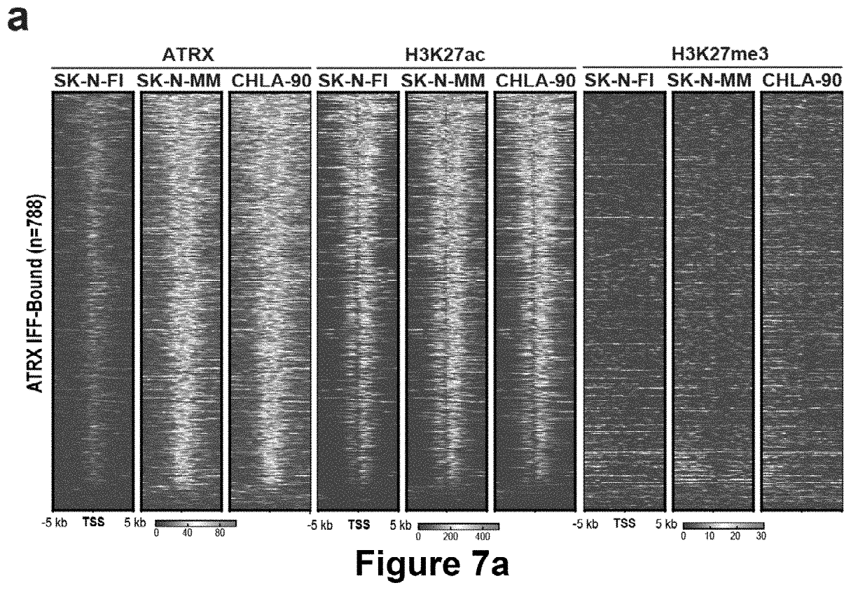

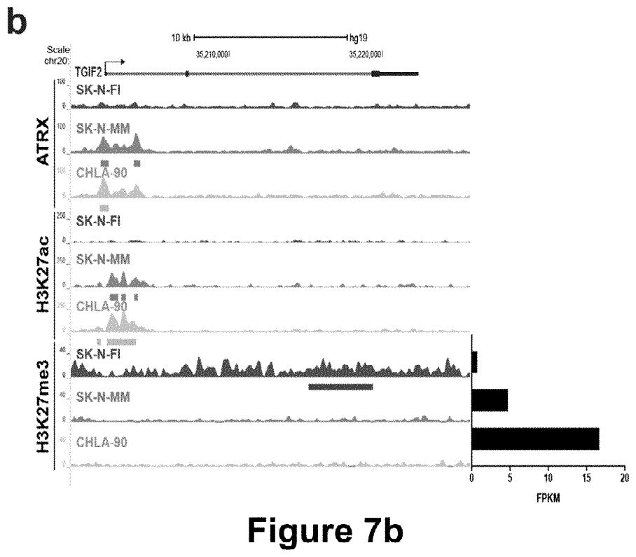

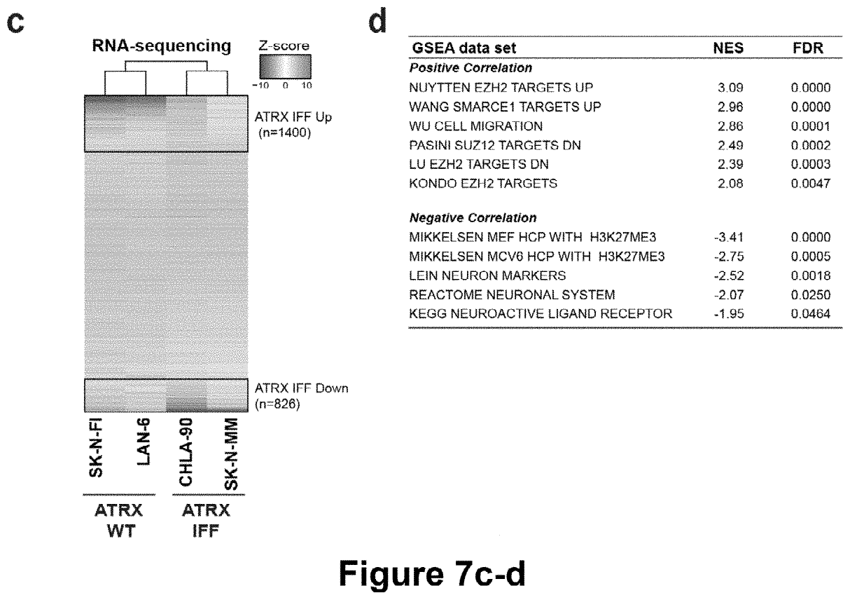

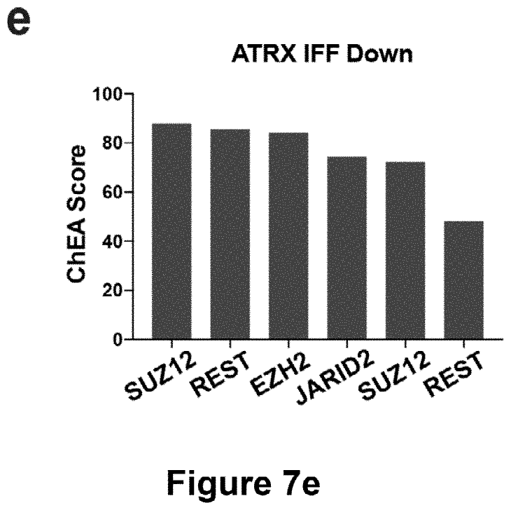

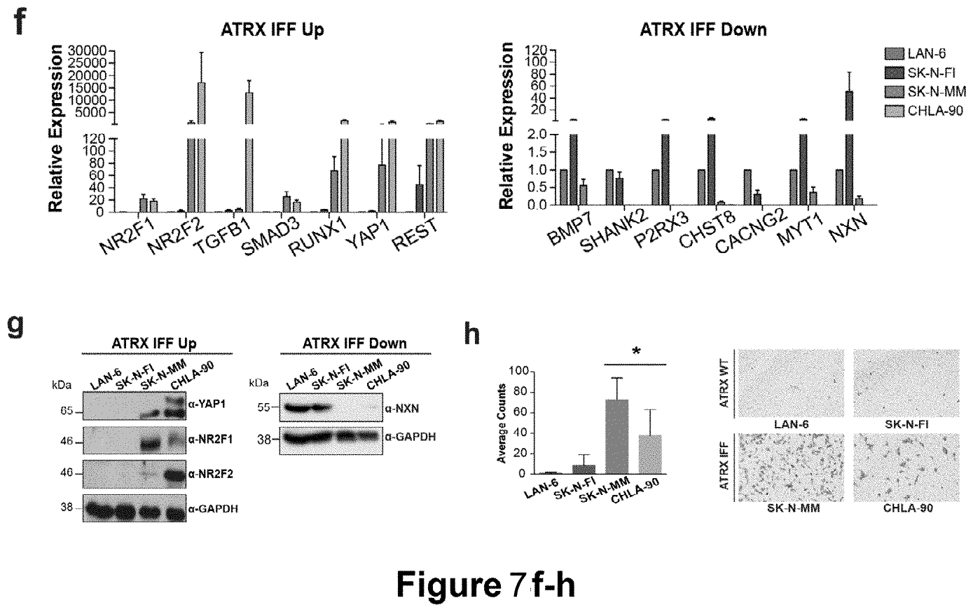

The inventors probed the transcriptome of LAN-6, SK-N-FI, SK-N-MM and CHLA-90 cells and found that ATRX IFF NB cell lines harbored a unique transcriptional program relative to ATRX WT cells, with differentially expressed genes (fold-change ≥8) in ATRX IFF cells displaying a correlation with PRC2 targets and anti-correlation with neuronal markers (FIG. 7 c-e).

FIG. 7 shows an integrated analysis of ChIP-seq and RNA-seq from NB cells and validation. (a) Heatmap of ATRX, H3K27ac, and H3K27me3 enrichment in the ATRX IFF-bound promoters, (promoters: TSS±2 kb). (b) UCSC Genome Browser snapshot of ATRX, H3K27ac, and H3K27me3 ChIP-seq at the TGIF2 locus (y axis=reads per kilobase per million reads). Significant peaks called by both MAC2 and annotated under each profile by colored bar (FDR=5×10⁻² for SKNFI, 5×10⁻³ for SKNMM, 5×10⁻⁴ for CHLA90 ATRX peaks; and 1×10⁻⁵ for all cell lines H3K27ac and H3K27me3 peaks). (c) Heatmap of Fragments Per Kilobase Million (FPKM) values from RNA-seq analysis, unsupervised hierarchical clustering. (d) Table summarizing Gene Set Enrichment Analysis (GSEA) terms associated with differentially expressed genes in ATRX IFF cells. (e) ChIP-seq Enrichment Analysis (ChEA) of ATRX IFF Down genes (n=826), ranked by ChEA combined score. ChEA is a tool that correlates input gene lists with ChIP-seq data sets to discover novel associations. (f) qRT-PCR analysis of selected ATRX IFF Up and ATRX IFF Down genes identified by RNA-seq. Relative expression normalized to GAPDH, mean n±SD (n=5). (g) Immunoblots for ATRX IFF Up genes YAP1, NR2F1, and NR2F2 and ATRX IFF Down gene N×N from whole cell extracts of NB cells. GAPDH used as a loading control. (h) Trans-well migration assay of NB cell lines. Migrated cells stained with crystal violet 18 hours post-plating. Images taken at 20× and cells quantified, mean n±SD (n=3). Statistical significance assessed using student's unpaired t-test comparing each ATRX IFF cell line to both WT (* indicates p<0.05).

The inventors next focused on the 1,400 “ATRX IFF Up” and 826 “ATRX IFF Down” genes (FIG. 1e, FIG. 7c). Gene ontology (GO) analysis demonstrated that ATRX IFF Up genes are involved in proliferation, adhesion, and migration, while ATRX IFF Down genes are implicated in synaptic functions and neuronal differentiation (FIG. 1f, g). These differentially expressed genes were validated at both the mRNA and protein level (FIG. 1h, FIG. 7f, g). Further, ATRX IFF NB cells presented a more migratory phenotype than WT cells in functional assays. (FIG. 7h).

After integrating the RNA-seq and ChIP-seq data, the inventors discovered that ATRX IFF Up genes were enriched for ATRX IFFs and H3K27ac, and were depleted of H3K27me3 (FIG. 1i, FIG. 7b). Significantly, the inventors also noted the involvement of the transcriptional repressor REST (FIG. 1h-j), which mediates silencing of neuronal genes in non-neuronal lineages by binding repressor element-1 (RE-1) motifs and recruiting co-repressors. Upon differentiation, REST expression declines as neural progenitors progress to terminal neurons. In contrast, REST is transcriptionally upregulated in stage 4 MYCN non-amplified NB and linked to proliferation and migration of glioblastoma cells.

The inventors further demonstrated that ATRX IFF proteins are depleted from H3K9me3-enriched chromatin, and instead, occupy active genomic elements associated with a unique gene expression signature, including the expression of REST and concomitant downregulation of its targets (FIG. 1g, h).

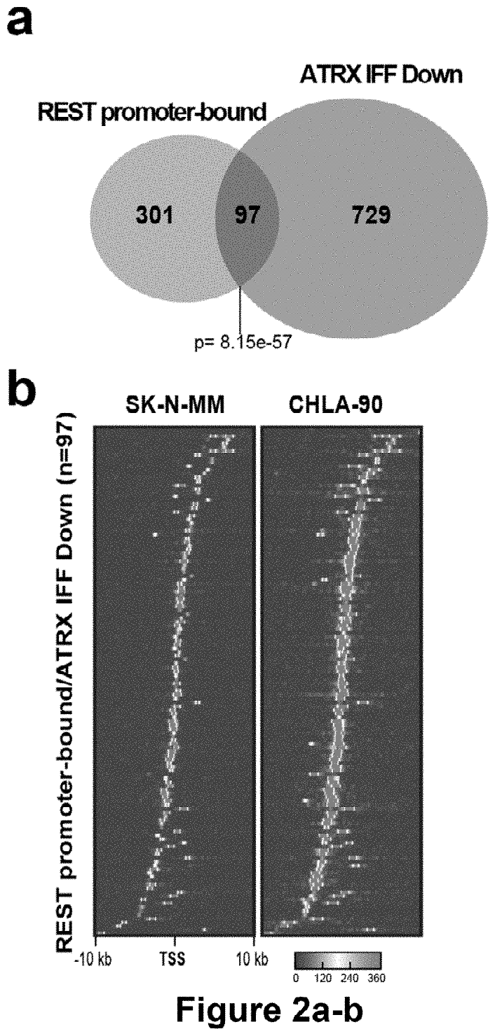

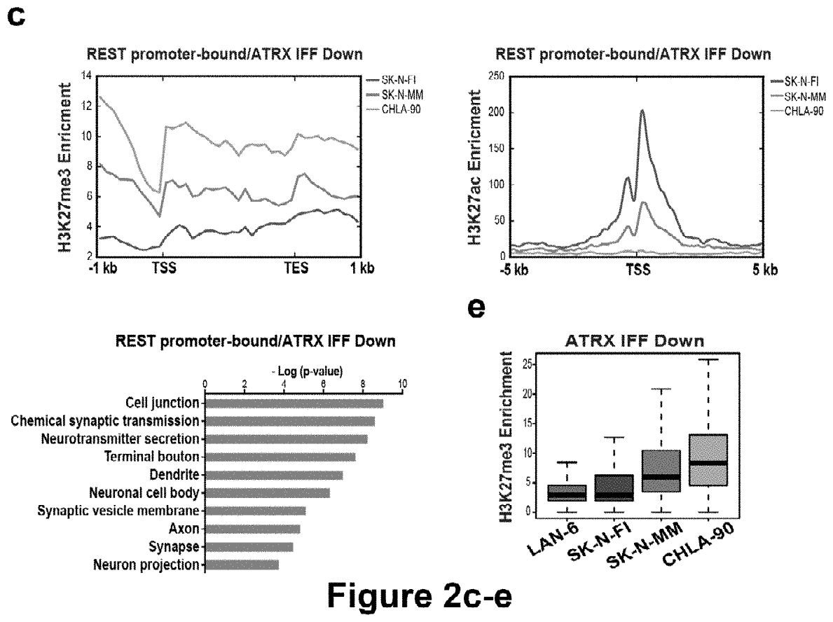

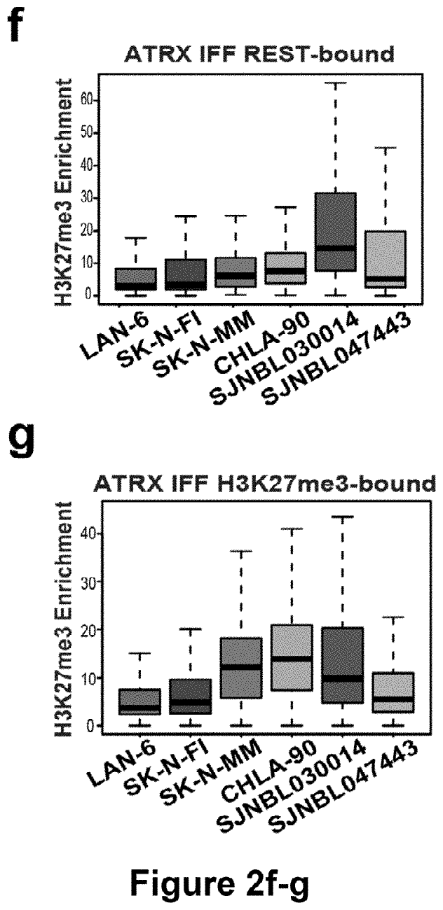

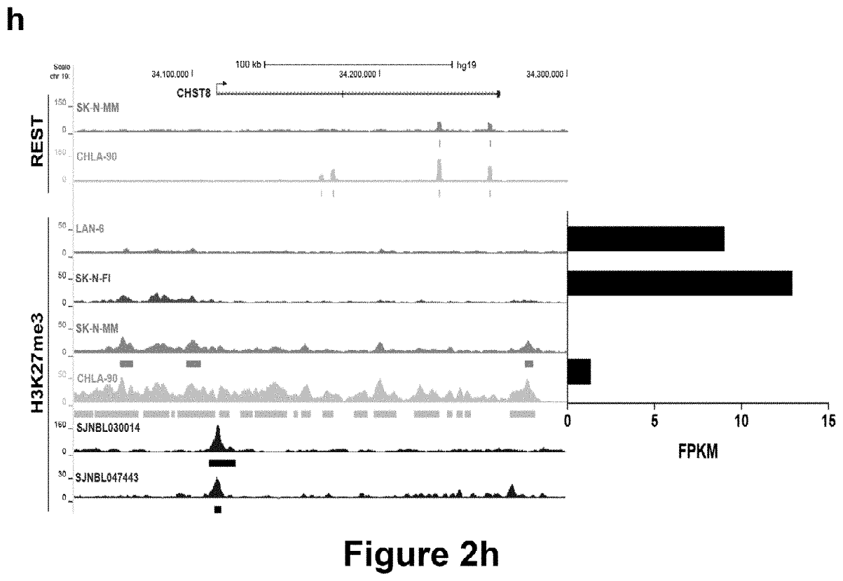

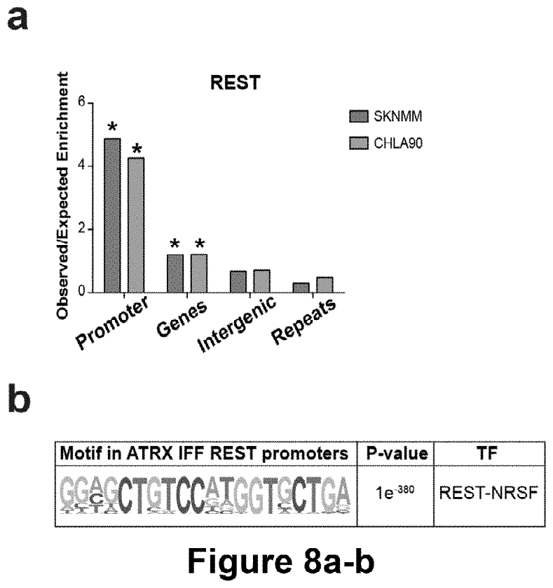

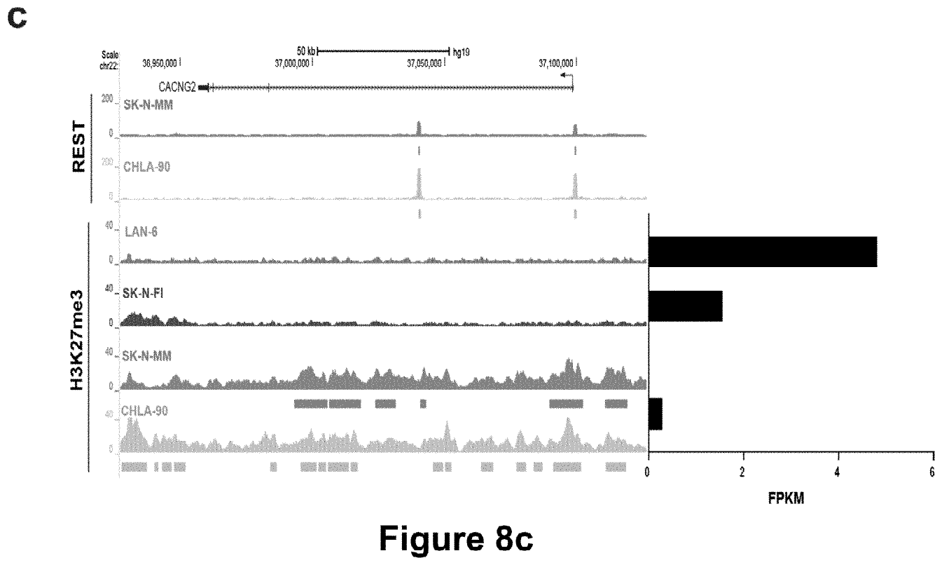

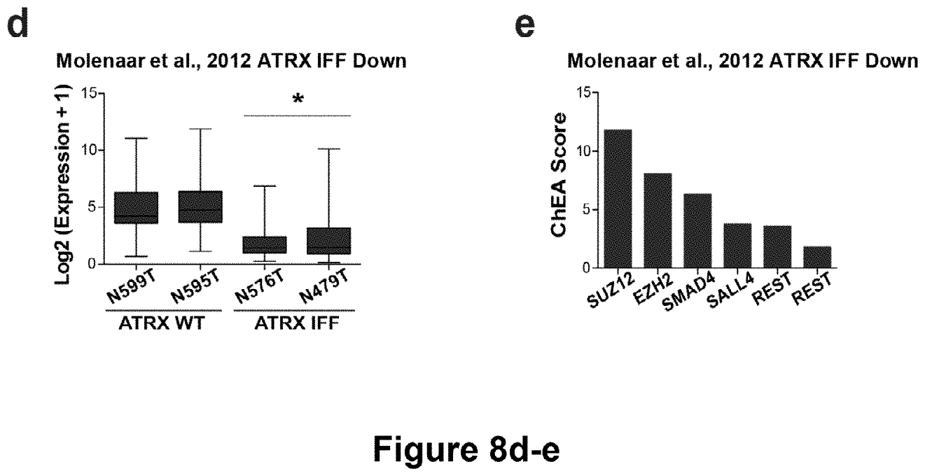

Because of the neuronal differentiation signature identified among ATRX IFF Down genes (FIG. 1e, f, FIG. 7e), coupled to robust levels of REST protein in ATRX IFF NB cells (FIG. 1h), the inventors performed REST ChIP-seq in SK-N-MM and CHLA-90. They discovered REST peaks, primarily in promoters and gene bodies (FIG. 8a). The inventors called the REST-bound promoters common to both ATRX IFF lines (n=398), and identified the REST DNA binding motif (FIG. 8b), as well as a significant overlap of REST-bound promoters with ATRX IFF Down genes CREST promoter-bound/ATRX IFF Down′, n=97) (FIG. 2a, b). Indeed, the inventors determined that ˜20% of ATRX IFF Down genes were REST-occupied in the promoter and/or gene body. The inventors further discovered that REST promoter-bound/ATRX IFF Down genes harbored greater H3K27me3, possessed reduced H3K27ac (FIG. 2c FIG. 8c), and were highly enriched for terminal neuronal differentiation terms annotated by GO (FIG. 2d).

FIG. 8 shows ChIP-seq analysis of REST in NB cells. (a) Observed over expected random distribution of significant REST peaks in chromatin state categories in ATRX IFF cells. Statistical significance assessed using hypergeometric test (* indicates p<0.01). (b) REST motif enriched at REST-bound promoters (n=398) analyzed using HOMER. (c) UCSC Genome Browser snapshot of REST and H3K27me3 ChIP-seq at the CACNG2 locus (y axis=reads per kilobase per million reads). Significant peaks called by MACS2 (REST) and both MAC2 and SICER (H3K27me3) annotated under each profile by colored bar (FDR=5×10⁻² for SK-N-MM and CHLA-90 REST peaks; and 1×10⁻⁵ for all cell lines H3K27me3 peaks). (d) Box-plots of expression levels in downregulated genes (n=180) in ATRX IFF NB tumors. Statistical significance assessed using student's unpaired t-test comparing each ATRX IFF patient compared to both WT (* indicates p<0.05). (e) ChIP-seq Enrichment Analysis (ChEA) of genes in (j), ranked based on ChEA combined score.

The inventors also mined NB patient microarray data and compared ATRX IFF NB samples for which data was available (annotated N576T and N479T) to ATRX WT tumors matched for age, gender, and MYCN status. Genes that had lower expression (ATRX IFF Down) in ATRX IFF NB samples were predicted as targets of PRC2 and REST using ChIP-seq enrichment analysis (ChEA) (FIG. 8d, e), a tool that correlates input gene lists with ChIP-seq data sets to discover novel associations. These data corroborated the role of REST in suppressing expression of neurogenesis genes in ATRX IFF NB.

FIG. 2 shows that ATRX IFF proteins in NB cells exhibit REST and H3K27me3 occupancy at repressed neuronal differentiation genes. (a) Venn diagram comparing REST-bound promoters (n=398) and Down genes (n=826) in ATRX IFF NB cells. Statistical significance assessed using hypergeometric distribution test. (b) Heatmap of REST enrichment in REST promoter-bound/ATRX IFF down promoters, (promoters: TSS±10 kb). (c) Metagene analysis of H3K27me3 (TSS to TES, ±1 kb) and H3K27ac (TSS±5 kb) enrichment at REST promoter-bound/ATRX IFF Down promoters (n=97). (d) Gene ontology (GO) analysis of REST promoter-bound/ATRX IFF Down genes. Enriched terms ranked by most significant p-value. (e) Boxplots of H3K27me3 enrichment in ATRX IFF Down genes in NB cell lines. Statistical significance assessed using one-way ANOVA and summarized in Table 2. (f) Boxplots of REST enrichment at ATRX IFF REST-bound genes (n=800). Statistical significance assessed using one-way ANOVA and summarized in Table 3. (g) Boxplots of H3K27me3 enrichment in exclusively ATRX IFF H3K27me3-bound genes (n=780) in ATRX IFF NB cells and NB patients. Statistical significance assessed using one-way ANOVA and summarized in Table 2. (h) UCSC Genome Browser snapshot of REST and H3K27me3 ChIP-seq at the CHST8 locus (y axis=reads per kilobase per million reads). Significant peaks called by MACS2 (REST) and both MAC2 and SICER (H3K27me3) annotated under each profile by colored bar (FDR=5×10⁻² for REST peaks; 1×10⁻⁵ for all cell lines, 5×10⁻² for SJNBL047443 and 1×10⁻¹⁵ for SJNBL030014 H3K27me3 peaks).

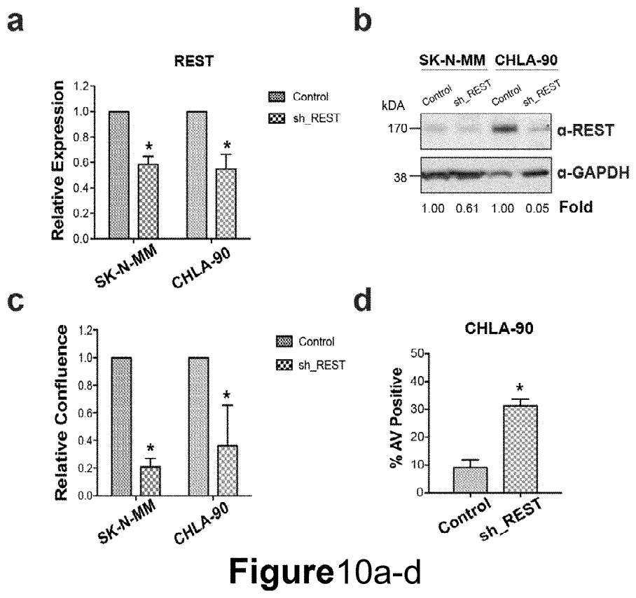

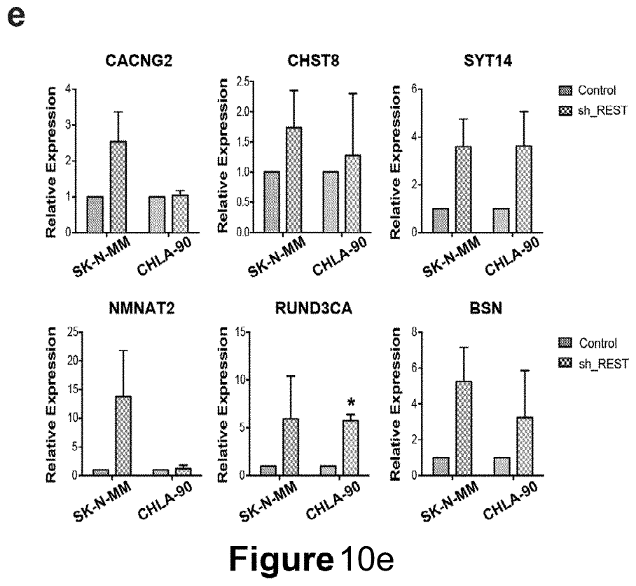

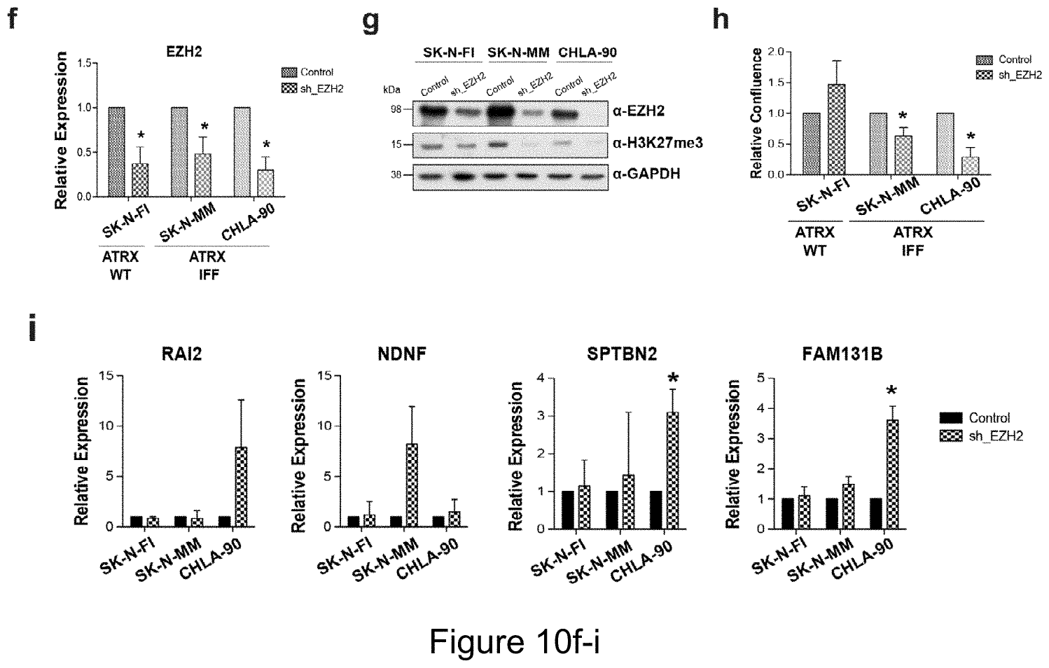

The inventors also analyzed H3K27me3-enriched genes and/or promoters in ATRX WT and IFF cells. Analysis of common significant peaks in SK-N-MM and CHLA-90 revealed that ATRX IFF NB had increased enrichment of H3K27me3 in promoters, intergenic regions and repeats compared to WT ATRX NB (FIG. 8a, b). The inventors showed that ATRX IFF NB cells exhibited increased H3K27me3 at Down genes (FIG. 2e). These discoveries were further revealed using H3K27me3 ChIP-seq data from two ATRX IFF NB patient tumor samples: a metastasis collected at autopsy from the retroperitoneum (SJNBL030014; IFF of exons 1 to 11) (FIG. 10c-e) and a PDX generated from a recurrent paraspinal NB (SJNBL047443; IFF of exons 2-11).

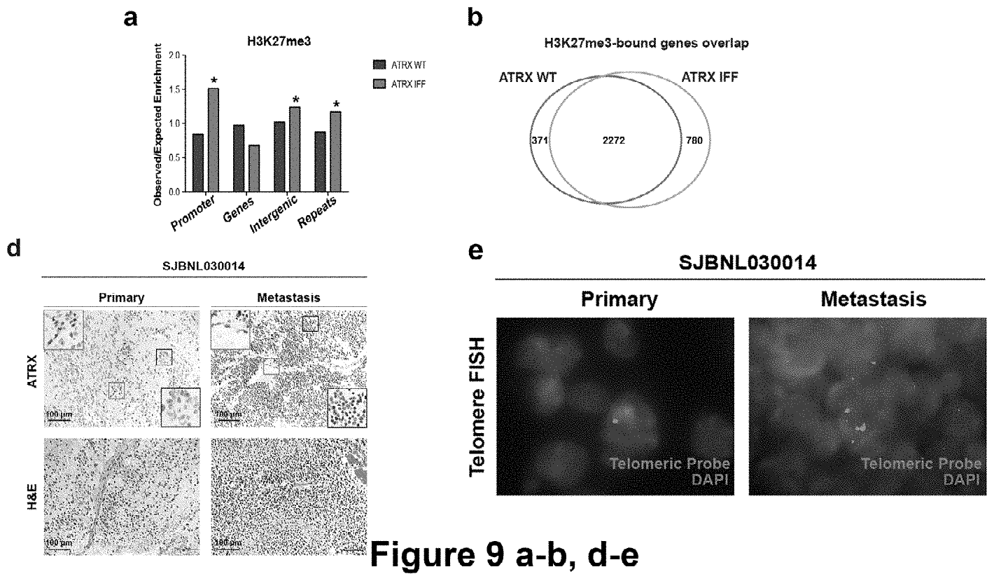

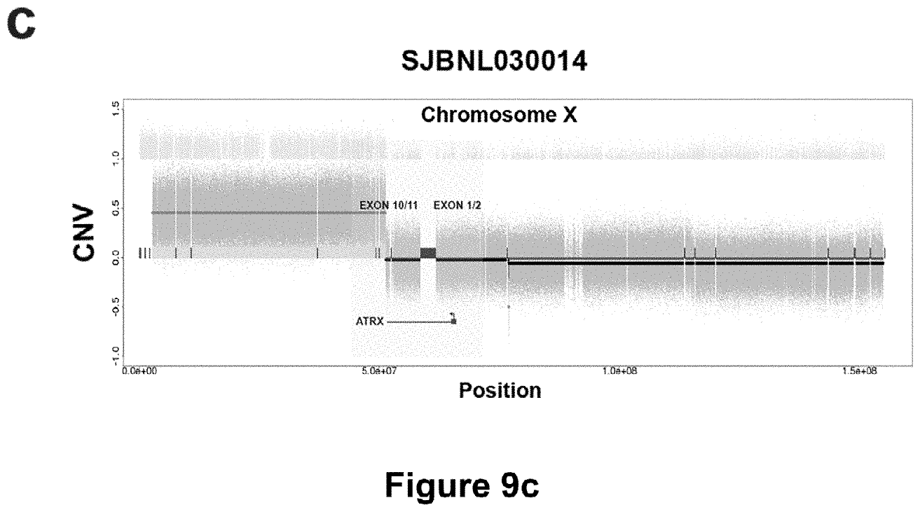

FIG. 9 shows ChIP-seq analysis of H3K27me3 in NB cells and characterization of ATRX IFF NB patient tumors. (a) Observed over expected random distribution of significant H3K27me3 peaks in chromatin state categories in ATRX WT vs IFF cells. (b) Venn diagram comparing H3K27me3-bound genes at promoters and/or gene bodies exclusively in both ATRX WT and ATRX IFF NB cells. (c) Wiggle plot of copy number variation (CNV) of Chromosome X from ATRX IFF NB patient. ATRX IFF indicated. (d) Immunostaining (top) for ATRX in primary and metastatic NB tissues. Black insets (bottom right) show nuclei magnified 250× and red inset (top left) shows ATRX positive control endothelial cells. H&E staining (bottom) of NB tissue. Images taken at 20×, scale bar 100 μm. (e) Image of ATRX IFF NB patient cells hybridized with telomere FISH probe (red) and stained with DAPI to visualize the nucleus (blue).

Both ATRX IFF tumor specimens exhibited enrichment of H3K27me3 at the REST- and H3K27me3-enriched genes called in ATRX IFF cell lines (FIG. 2f, g), such as the REST target CHST8 (FIG. 2h). Thus, the repression of neuronal specification genes is reinforced by REST occupancy at a subset of H3K27me3-decorated genes.

In neural stem cells, knockdown of REST broadly upregulates neuronal gene expression, promoting differentiation. The inventors assessed REST knockdown in ATRX IFF NB cells and observed decreased proliferation in both ATRX IFF NB cell lines (FIG. 10a-c). REST knockdown in CHLA-90 cells, which express the protein more abundantly (FIG. 1h), also induced apoptosis (FIG. 10d). Importantly, the inventors discovered the derepression of REST targets in knockdown vs. control cells, demonstrating that REST acts as a transcriptional repressor at particular neurogenesis genes (FIG. 10e). Because the inventors also identified H3K27me3 enrichment at ATRX IFF Down genes (FIG. 2e), they also performed EZH2 knockdown. This generated a similar phenotype as with REST knockdown-reduced proliferation and upregulation of corresponding genes in ATRX IFF NB cells (FIG. 10f-i).

FIG. 10 shows that REST and EZH2 knockdown reduces proliferation NB cells positive for ATRX IFF. (a) qRT-PCR of REST knockdown in NB cells. Relative expression normalized to RPL0, mean n±SD (n=3). (b) Immunoblots for REST in control and knockdown NB cells. GAPDH used as a loading control. (c) Proliferation of control and REST knockdown NB cells at day 9 after puromycin selection. Proliferation evaluated using the Incucyte™ Life-Cell Imaging System. Percent confluence normalized to DMSO for each condition and plotted at day 9, values are mean n±SD (n=3). (d) Annexin V (AV) staining of control and REST knockdown NB cells. Percent AV positive cells plotted, values are mean n±SD (n=3). Statistical significance assessed using student's unpaired t-test with each cell line compared to control cells (* indicates p<0.05). (e) qRT-PCR of REST target genes in NB cells day 3 after puromycin selection. Relative expression normalized to RPLO, mean n±SD (n=2). Statistical significance assessed using student's unpaired t-test with each cell line compared to control cells (* indicates p<0.05). (f) qRT-PCR of EZH2 knockdown in NB cells. Relative expression normalized to GAPDH, mean n±SD (n=3). Statistical significance assessed using student's unpaired t-test with each cell line compared to control cells (* indicates p<0.05). (g) Immunoblots for EZH2 and H3K27me3 in control and knockdown NB cells. GAPDH used as a loading control. (h) Proliferation of control and EZH2 knockdown NB cells at day 12 after puromycin selection. Proliferation evaluated using the Incucyte™ Life-Cell Imaging System. Percent confluence normalized to DMSO for each condition and plotted at day 12, values are mean n±SD (n=3). Statistical significance assessed using student's unpaired t-test with each cell line compared to control cells (* indicates p<0.05). (i) qRT-PCR analysis in control and EZH2 knockdown NB cells of ATRX IFF H3K27me3-bound genes at day 5 after puromycin selection. Relative expression normalized to GAPDH, mean n±SD (n=2).

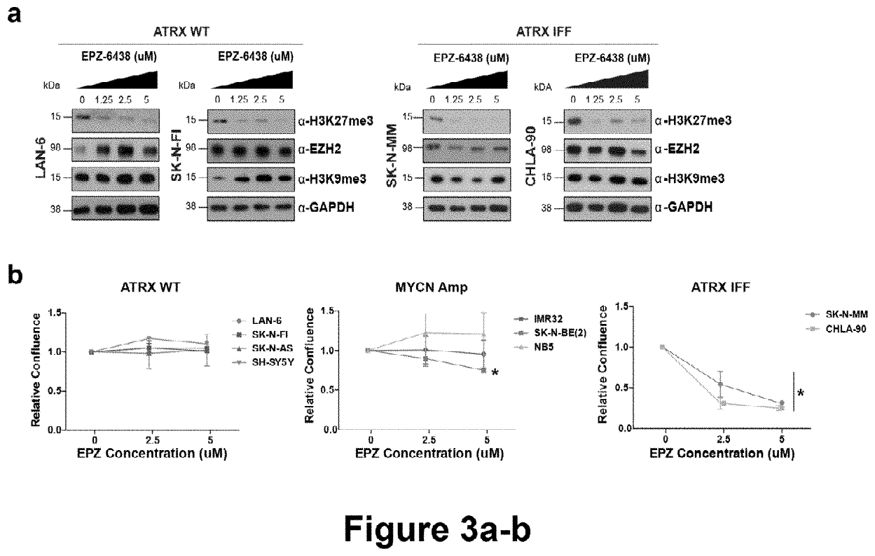

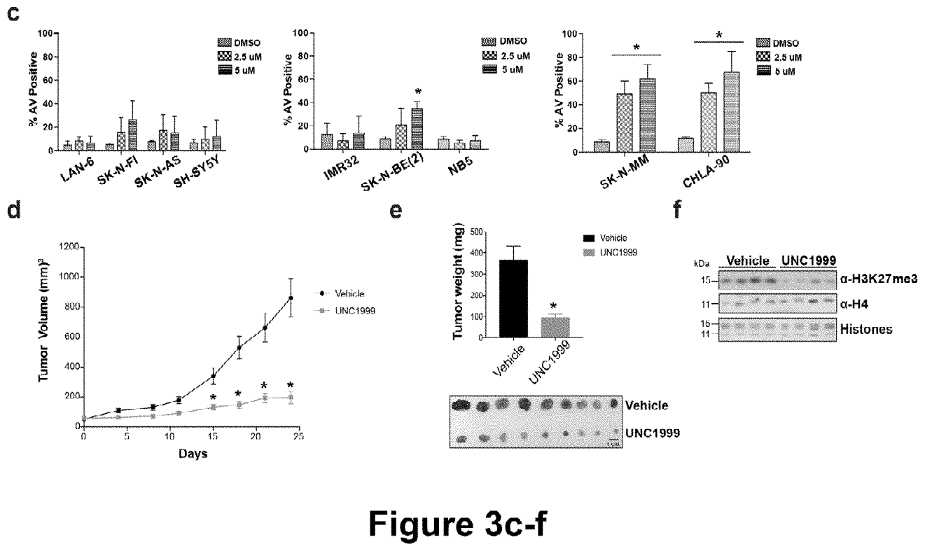

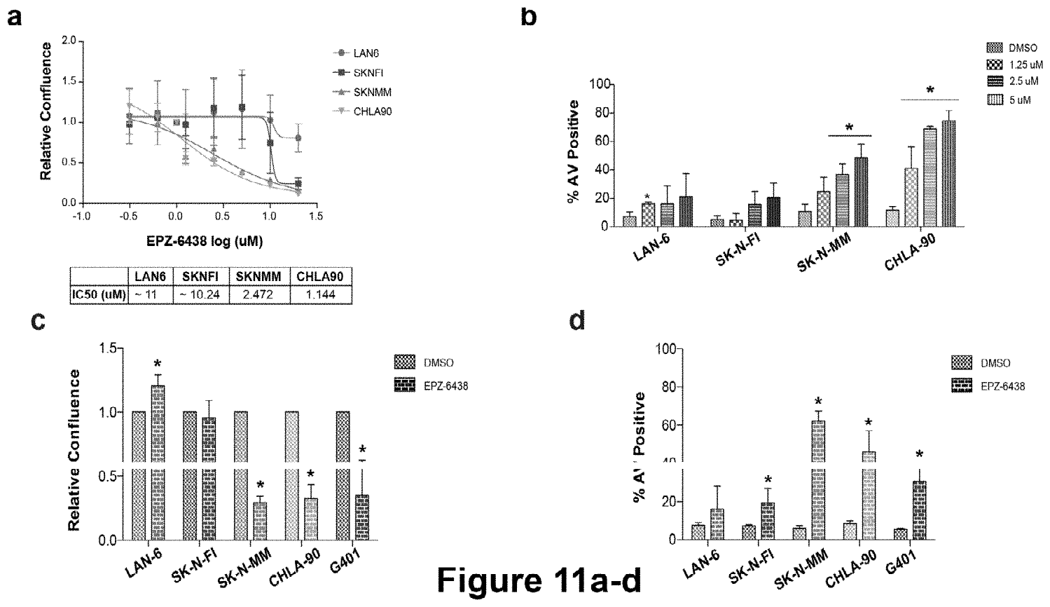

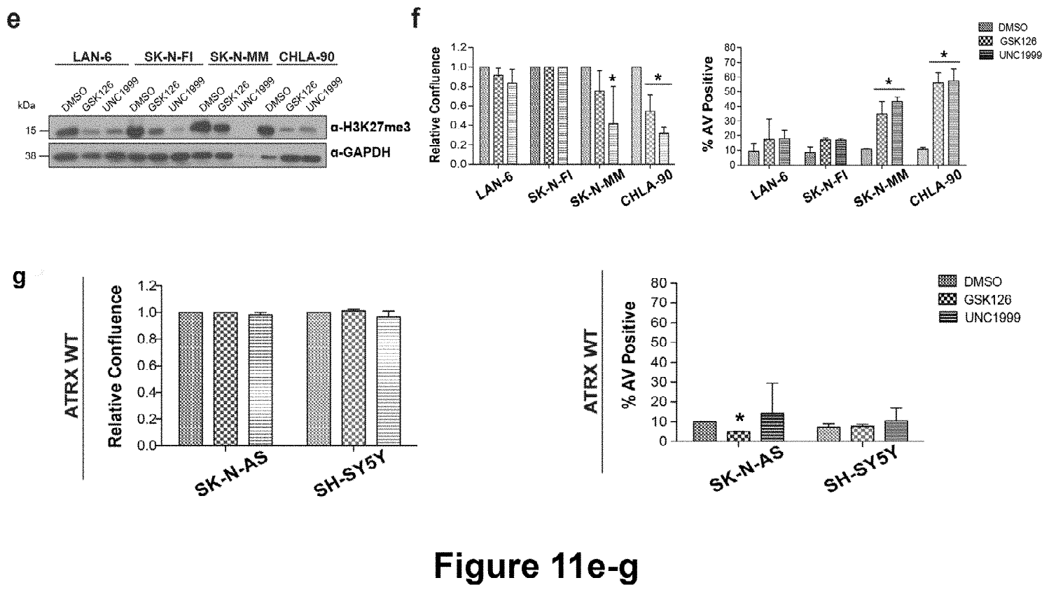

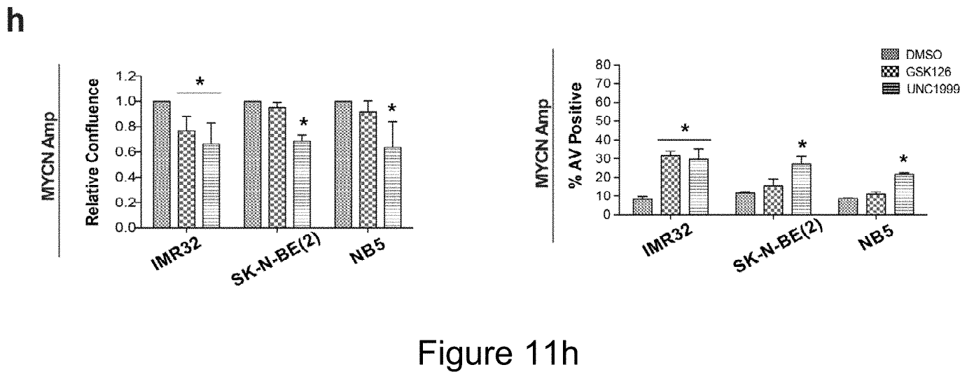

The inventors discovered that ATRX IFF NB cells can be targeted with EZH2 inhibitors (EZH2i) to alleviate H3K27me3-mediated silencing of neuronal genes, whether REST-dependent or not. The inventors utilized the clinical EZH2i, EPZ-6438 (Tazemetostat), and treated a panel of NB cells with increasing drug concentration. The inventors determined that EPZ-6438 efficiently depleted H3K27me3, while not affecting H3K9me3 (FIG. 3a). Increasing dosage of EPZ-6438 resulted in decreased proliferation, followed by increased apoptosis in ATRX IFF NB cells (at comparable concentrations as G401, an EZH2i-sensitive SNF5-mutant malignant rhabdoid tumor (MRT) cell line) (FIG. 3b, c, FIG. 11 a-d). ATRX IFF NB cells were particularly sensitive compared to other NB subtypes (5-10 fold; FIG. 11a). Some MYCN-amplified NB cell lines showed mild sensitivity (FIG. 3b, c). The use of two additional EZH2i, GSK126²⁶ and UNC1999 achieved similar results (FIG. 11e-h).

FIG. 3 shows that EZH2 inhibition in NB cells positive for ATRX IFF leads to cell death and tumor regression. (a) Immunoblots for H3K27me3, EZH2, and H3K9me3 from whole-cell extracts of NB cells treated with DMSO or EPZ-6438 (2.5 or 5 μM) for 7 days. GAPDH used as a loading control. (b) Proliferation curves for NB cell lines treated with DMSO or EPZ-6438 (2.5 or 5 μM) over 12 days. Percent confluence normalized to DMSO for each condition and plotted at day 12. Values normalized mean n±SD (n=3). Statistical significance assessed using student's unpaired t-test with each cell line compared to DMSO (* indicates p<0.05). (c) Annexin V (AV) staining of NB cells treated with DMSO or EPZ-6438 (2.5 or 5 μM) for 12 days. Percent AV positive cells plotted, values are mean n±SD (n=3). Statistical significance assessed using student's unpaired t-test with each cell line compared to DMSO (* indicates p<0.05). (d) Tumor volume measurements of CHLA-90 cells subcutaneously injected into immunocompromised mice. Mice were treated with vehicle or UNC1999 at 300 mg/kg of body weight for 25 days, values mean n±SEM (n=9). Statistical significance assessed using student's unpaired t-test compared to vehicle (* indicates p<0.001). (e) Tumor weight measurement from xenografts collected in (d), values mean n±SEM (n=9). Images of excised tumors underneath (* indicates p<0.001). (f) Immunoblots for H3K27me3 from acid extracted histones derived from vehicle and treated xenografts. H4 and histones used as loading controls.

FIG. 11 shows that EZH2i increase apoptosis in ATRX IFF NB cells. (a) Proliferation curves of NB cells treated with increasing concentration of EPZ-6438. Proliferation evaluated using the INCUCYTE Life-Cell Imaging System. Percent confluence normalized to DMSO for each condition and plotted at day 12, values mean n±SD (n=2). IC50 for each cell line calculated in Prism. (b) Annexin V (AV) staining of NB cell lines treated with increasing concentration of EPZ-6438 at day 12. Percent AV positive cells plotted, values are mean n±SD (n=3). Statistical significance assessed using student's unpaired t-test with each cell line compared to DMSO (* indicates p<0.05). (c) Proliferation curves comparing NB cell lines to EZH2i-sensitive malignant rhabdoid tumor cell line G401. Cells treated with DMSO or EPZ-6438 (5 μM) for 12 days, values mean n±SD (n=3). Proliferation assessed in the same manner as (a). Statistical significance assessed using student's unpaired t-test with each cell line compared to DMSO (* indicates p<0.05). (d) Annexin V (AV) staining of NB cell lines and G401 treated with DMSO or EPZ-6438 (5 μM) at day 12, values are mean±SD (n=3). AV staining assessed in the same manner as (b). (e) Immunoblots for H3K27me3 of whole cell extracts from NB cell lines treated with DMSO, GSK126 or UNC1999 (504) for 10 days. GAPDH used as a loading control. (f), (g), and (h) Proliferation curves (left panel) for NB cell lines treated with DMSO, GSK126 or UNC1999 (504) for 12 days, values are mean n±SD (n=3). Proliferation assessed in the same manner as (a). Annexin V (AV) staining (right panel) of NB cell lines treated with DMSO, GSK126 or UNC1999 (504) at day 12, values are mean n±SD (n=3). AV staining assessed in the same manner as (b).

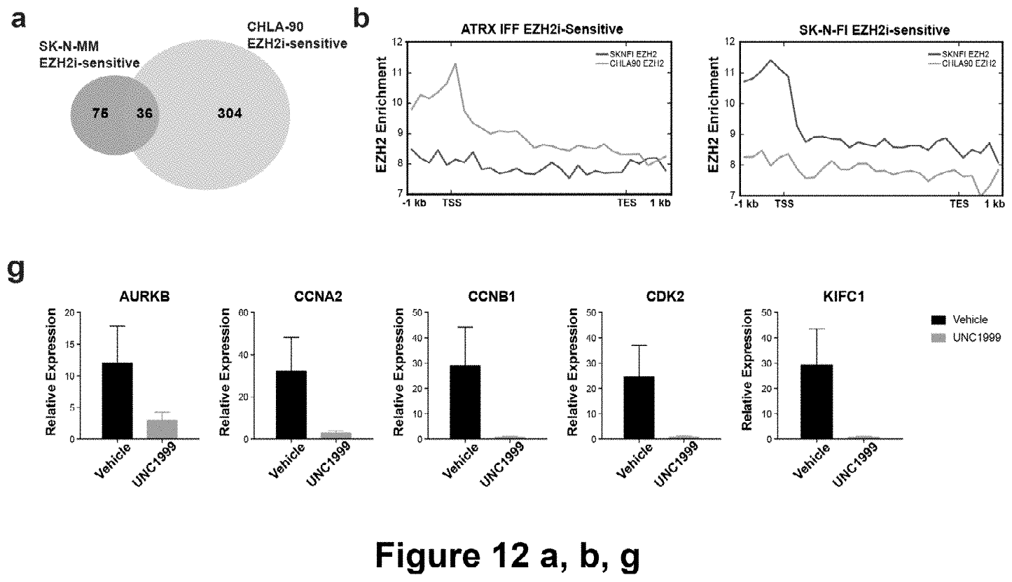

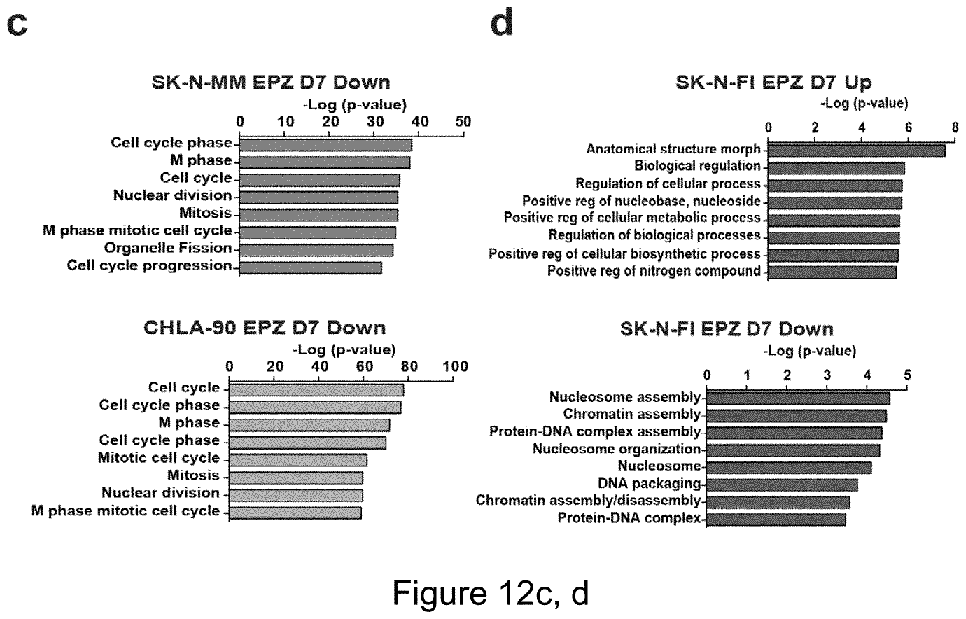

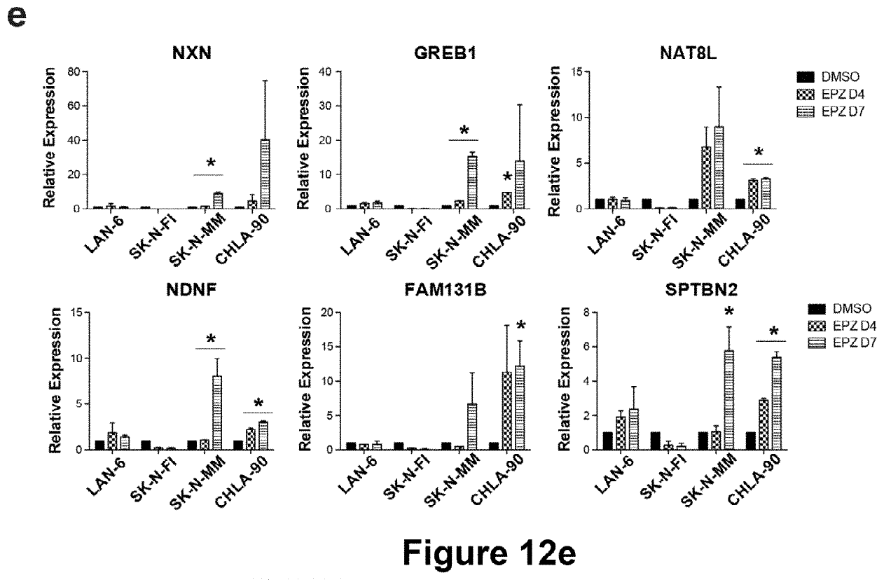

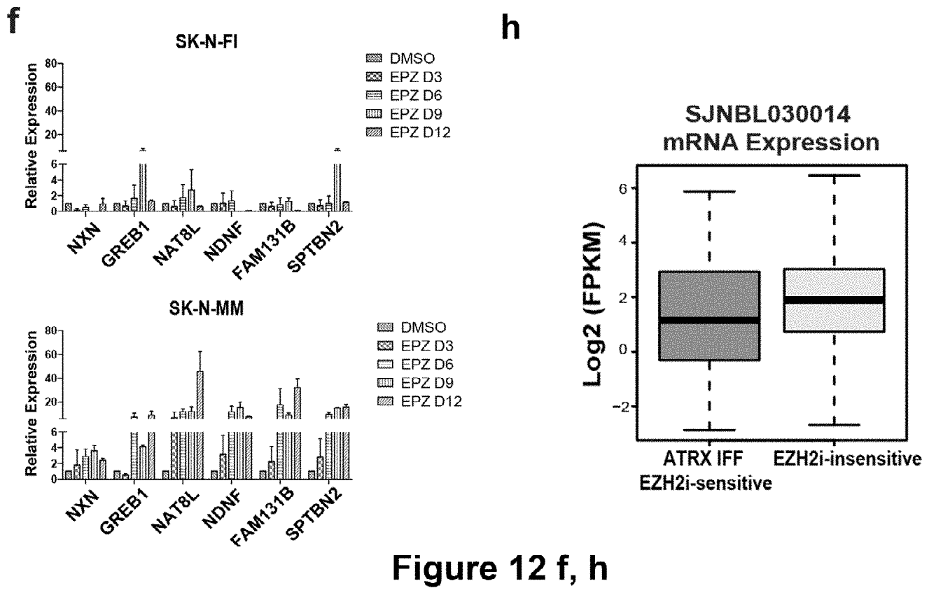

The inventors assessed EPZ-6438 treatment of NB cells over a time course and discovered reduced levels of H3K27me3, correlating with diminished proliferation and increased apoptosis, exclusively in ATRX IFF NB (FIG. 12a-c). Further, NB cells grown as multicellular tumorspheres revealed a striking decrease in the percentage of live cells in EPZ-6438-treated ATRX IFF NB tumorspheres (FIG. 12d-f). Importantly, CHLA-90 xenografts treated with UNC1999 for 25 days displayed a significant reduction in tumor growth and volume compared to vehicle-treated tumors, reinforcing the sensitivity to EZH2i in vivo (FIG. 3d-f).

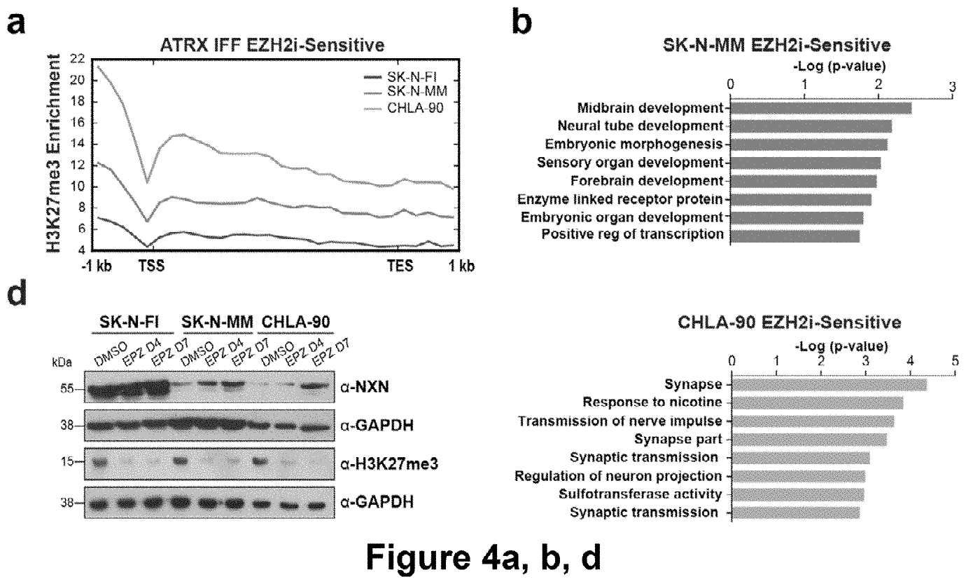

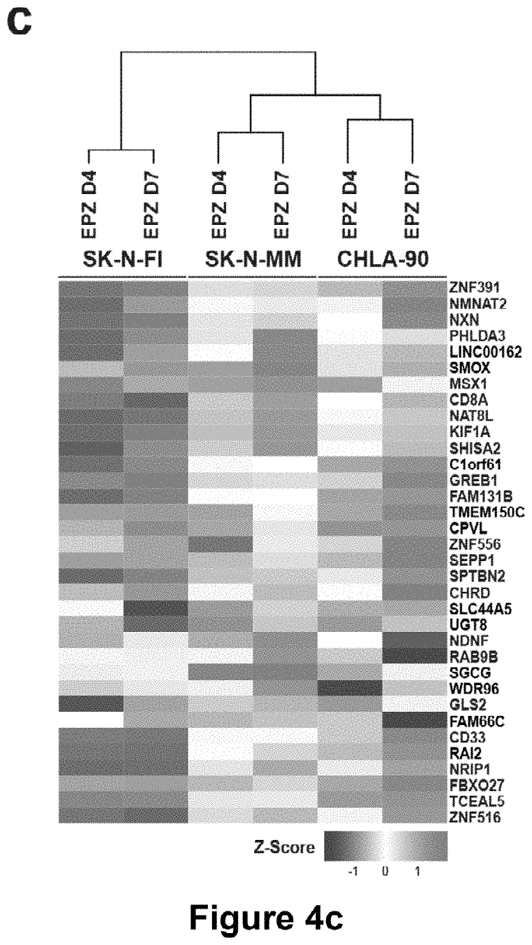

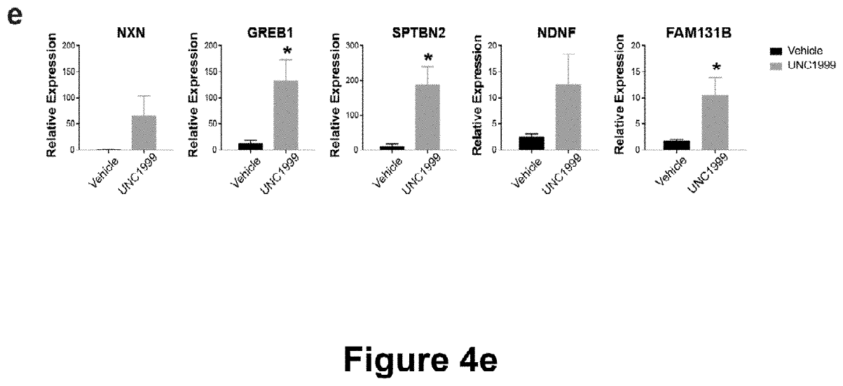

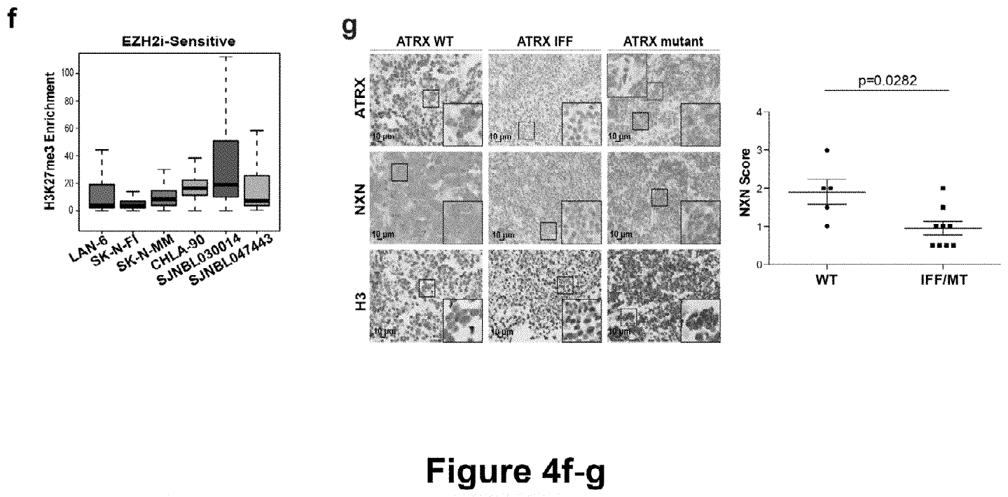

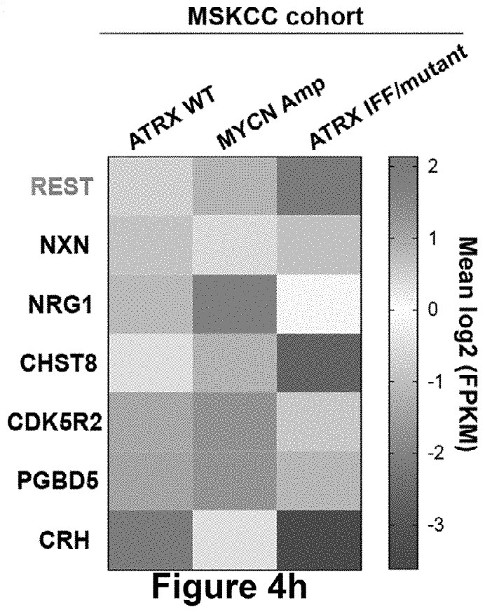

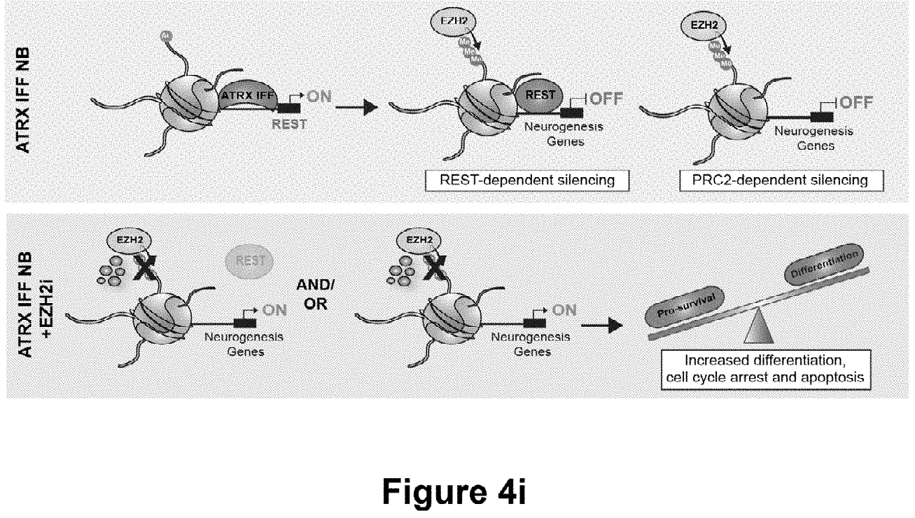

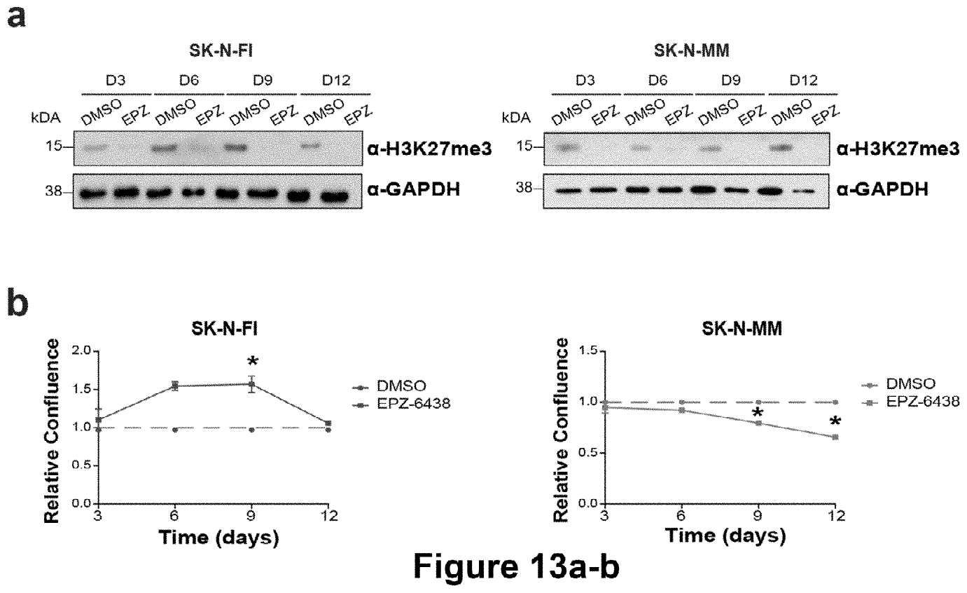

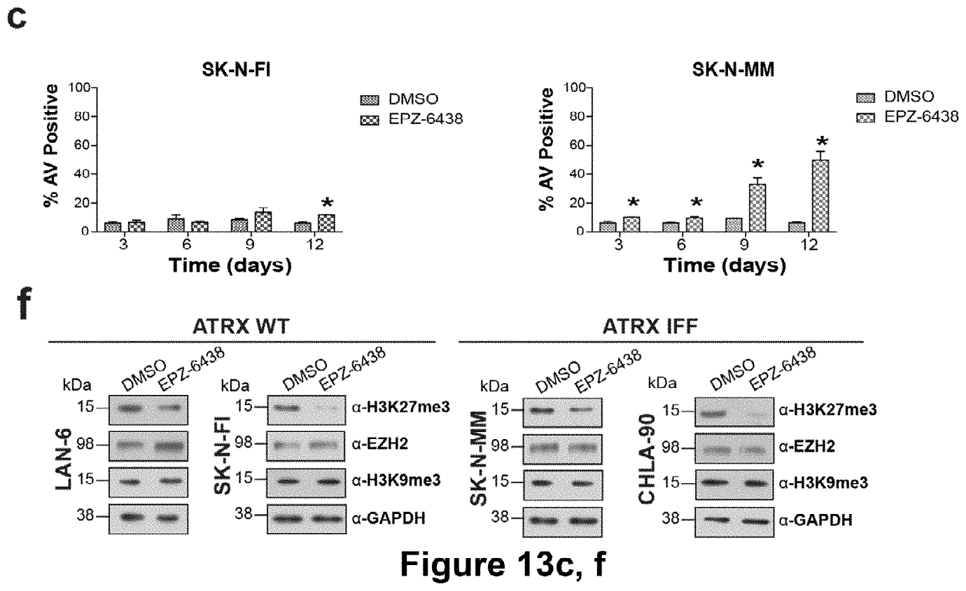

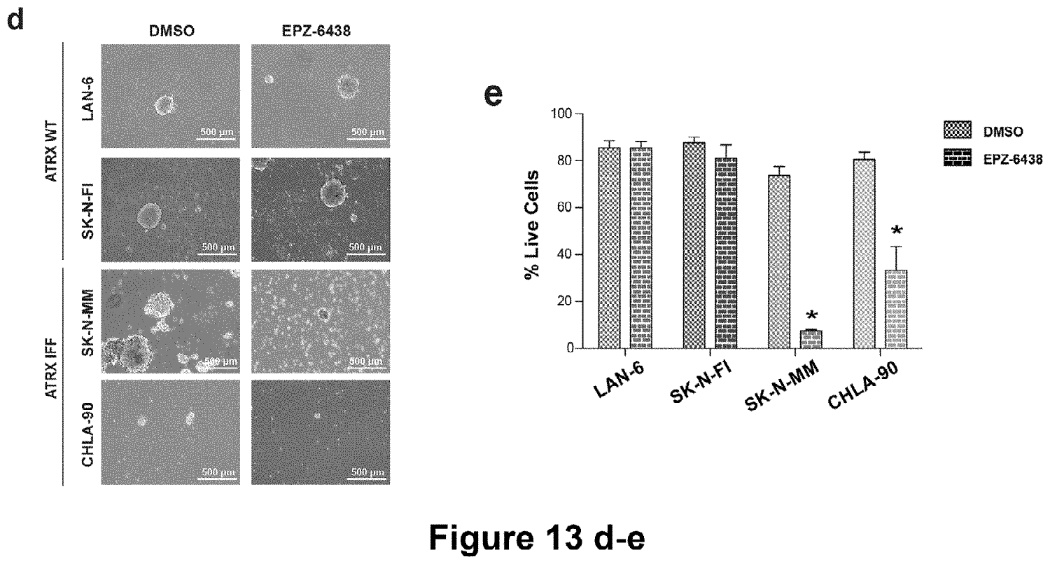

The inventors tested the expression profile of ATRX WT (SK-N-FI) and ATRX IFF (SK-N-MM and CHLA-90) cells treated with EPZ-6438 for 4 and 7 days. Stringent criteria were utilized to focus on genes that were i) exclusively upregulated in EZH2i-treated vs. DMSO-treated cells, and ii) had significant H3K27me3 enrichment in untreated cells. “EZH2i-sensitive” genes were pooled together from D4 and D7 in each cell line and confirmed to be on-target by H3K27me3 and EZH2 enrichment (FIG. 4a, FIG. 13a, b, Table 4). EZH2i-sensitive genes were implicated in brain development and neuronal processes by GO analysis with a common overlap of 36 neurogenesis genes (FIG. 4b, c, FIG. 13a, Table 4).

FIG. 13 shows RNA-seq analysis and validation of EZH2i-treated NB cells. (a) Venn diagram comparing overlap of genes that are i) upregulated in SKNMM and CHLA90 over DMSO treated cells pooled from day 4 and day 7 EPZ-6438 (5 μM)-treated cells and ii) H3K27me3-bound gene promoters in control cells. (b) Metagene analysis of EZH2 enrichment for SK-N-FI and CHLA-90 at ATRX IFF EZH2i-sensitive genes (left, n=415) and SKNFI EZH2i-sensitive genes (right, n=305), (TSS to TES, ±1 kb). (c) Gene ontology (GO) analysis of genes downregulated in EPZ-6438-treated ATRX IFF cells at day 7 over DMSO-treated cells. Enriched terms ranked by most significant p-value. (d) Gene ontology (GO) analysis of genes upregulated/downregulated in EPZ-6438-treated ATRX WT cell line SKNFI at day 7 over DMSO-treated cells. Enriched terms ranked by most significant p-value. (e) qRT-PCR analysis of EZH2i-sensitive genes. Relative expression normalized to GAPDH, mean n±SD (n=3). (f) qRT-PCR analysis of ATRX EZH2i-sensitive genes over several time points of treatment with DMSO or EPZ-6438 (5 μM) for 12 days. Relative expression normalized to GAPDH, mean n±SD (n=2). (g) qRT-PCR analysis of cell cycle genes downregulated by EZH2i in CHLA-90 vehicle and UNC1999 (300 mg/kg) treated xenografts. Relative expression normalized to GAPDH, mean n±SD (n=7). (h) Expression levels of EZH2i-sensitive genes versus EZH2i-insensitive genes in ATRX IFF NB patient tumor SJNBL030014.

The inventors discovered that approximately 10% of EZH2i-sensitive genes were in fact REST targets identified previously by ChIP-seq, indicative of concordance between REST and EZH2 function. The inventors further found that cell cycle and mitotic genes were significantly downregulated in ATRX IFF NB cells at D7 (FIG. 13c), consistent with a growth arrest prior to apoptosis (FIG. 3). In contrast, ATRX WT NB cells proliferated normally (or faster) when treated with EPZ-6438 (FIG. 3, FIG. 12b). Moreover, SK-N-FI cells did not show changes in expression of neurogenesis genes upon EPZ-6438 treatment (FIG. 4c), and were associated with nonspecific GO terms (FIG. 13d, Table 4).

The inventors examined RNA-seq data from ATRX WT, MYCN-amplified, and ATRX-altered patient tumors and found that REST expression was higher in ATRX IFF/mutant patients along with reduced expression of several EZH2i-sensitive REST target genes, indicating silencing of neuronal transcriptional programs and, in turn, potential sensitivity to EZH2i (FIG. 4h).

FIG. 4 shows that EZH2 inhibition in NB cells positive for ATRX IFF de-represses H3K27me3- and REST-bound neuronal differentiation genes. (a) Metagene analysis of H3K27me3 enrichment in indicated NB cell lines at ATRX IFF EZH2i-sensitive genes (n=415), (TSS to TES, ±1 kb). (b) Gene ontology (GO) analysis of genes upregulated in each ATRX IFF cell line. Enriched terms ranked by most significant p-value. (c) Heatmap of expression levels of EZH2i-sensitive genes common to both SK-N-MM and CHLA-90 (n=36) in all conditions. (d) Immunoblots for N×N and H3K27me3 in whole cell extracts from NB cell lines treated with DMSO and EPZ-6438 (5 μM) at day 4 and 7. GAPDH used as a loading control. (e) qRT-PCR analysis of EZH2i-sensitive genes in CHLA-90 vehicle and UNC1999 (300 mg/kg) treated xenografts. Relative expression normalized to GAPDH, mean n±SD (n=7). Statistical significance assessed using student's unpaired t-test with each cell line compared to control cells (* indicates p<0.05). (f) Box-plot of H3K27me3 enrichment in NB cell lines, ATRX IFF NB tumor and PDX, at EZH2i-sensitive genes. Statistical significance assessed using one-way ANOVA and summarized in Table 4. (g) IHC for ATRX and N×N in representative ATRX WT (MSKCC ID #7716), ATRX IFF (MSKCC ID #17473), and ATRX mutant (MSKCC ID #17252) NB tissue. Images taken at 40×; black insets (bottom right) show nuclei magnified 250× and red inset (top left) shows ATRX positive control endothelial cells in the ATRX mutant tumor. H3 staining used for tissue quality. Scale bar 10 ∞m. IHC scores for N×N (right). (h) Expression levels of REST and ATRX IFF EZH2i-sensitive/REST targets from RNA-seq analysis of MSKCC NB patient samples, (n=5 for each subtype). Mean log 2 Fragments Per Kilobase Million (FPKM) values for each subtype plotted. (i) Model of EZH2i mediated sensitivity in ATRX IFF NB. ATRX IFFs redistribute to the REST promoter. REST and/or EZH2-mediated silencing at neurogenesis genes in ATRX IFF NB promotes impaired differentiation and survival. These genes are transcriptionally unregulated upon EZH2i treatment prompting apoptosis.

FIG. 12 shows (a) Immunoblots for H3K27me3 of whole cell extracts from ATRX WT (SK-N-FI) and ATRX IFF (SK-N-MM) NB cell lines collected every 3 days during treatment with either DMSO or EPZ-6438 (5 μM) for 12 days total. GAPDH used as a loading control. (b) Proliferation curves over time for NB cell lines treated with DMSO or EPZ-6438 (5 μM) for 12 days, values are mean n±SD (n=2). Proliferation evaluated using the Incucyte™ Life-Cell Imaging System. Percent confluence normalized to DMSO for each condition and plotted at each time point, values mean n±SD (n=2). Statistical significance assessed using student's unpaired t-test with each cell line compared to DMSO, *indicates p<0.05. (c) Annexin V (AV) staining over time of NB cell lines treated with DMSO or EPZ-6438 (5 μM) for 12 days total, values are mean n±SD (n=2). Percent AV positive cells plotted for each time point, values are mean n±SD (n=2). Statistical significance assessed using student's unpaired t-test with each cell line compared to DMSO, * indicates p<0.05. (d) Brightfield images of neuroblastoma tumorspheres treated with DMSO or EPZ-6438 (5 μM) at day 12. Images taken at 10×, scale bar 500 μm. (e) Quantification of (d). Tumorspheres were trypsinized to single cells, stained with trypan blue and quantified. Values are mean n±SD (n=3). Statistical significance assessed using student's unpaired t-test with each cell line compared to DMSO, * indicates p<0.05. (f), Immunoblots for H3K27me3, EZH2, and H3K9me3 from whole-cell extracts of tumorspheres treated with DMSO or EPZ-6438 (5 μM) for 5 days. GAPDH used as a loading control.

### SEQUENCE INFORMATION

Whole-exome sequencing and CNV data was deposited in European Bioinformatics Institute (EBI) European Genome-phenome Archive (EGA), under accession code EGAS00001002507. All RNA-sequencing and ChIP-sequencing data was deposited in the National Center for Biotechnology Information (NCBI) Gene Expression Omnibus (GEO), under accession code GSE100148.

1. A therapeutic agent that decreases EZH2 activity in ATRX mutant cells.

2. The therapeutic agent of claim 1, wherein the ATRX mutant cells are ATRX IFF mutant cells.

3. The therapeutic agent of claim 2, wherein the ATRX mutant cells originated from neuronal cell lines.

4. (canceled)

5. A therapeutic agent that decreases REST (RE-1 Silencing Transcription Factor) production or activity in cells.

6. The therapeutic agent of claim 5 that comprises a nucleotide, chemical compound, protein, or combination thereof and a pharmaceutically acceptable carrier or diluent.

7. (canceled)

8. (canceled)

9. (canceled)

10. (canceled)

11. (canceled)

12. The therapeutic agent of claim 5 that decreases REST production or activity in ATRX mutant cancer cells.

13. A composition that comprises the therapeutic agent of claim 5.

14. A composition that comprises the therapeutic agent of claim 6.

15. (canceled)

16. Method of treating a disease in a subject by administering the therapeutic agent of claim 5.

17. Method of treating a disease in a subject by administering the therapeutic agent of claim 6.

18. (canceled)

19. A treatment method comprising: obtaining tissue samples from a subject; isolating nucleic acid sequences present in the tissue sample; screening for the presence of REST; comparing the REST levels to a predetermined amount; and administering to the subject a composition that decreases REST production or activity in a subject in need thereof.

20. (canceled)

21. (canceled)

22. (canceled)

23. (canceled)

24. (canceled)

25. (canceled)

26. (canceled)

27. The method of claim 19, wherein said therapeutic composition reduces the amount of REST below predetermined levels.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The method of claim 16, further comprising administering an EZH2 inhibitor or degrader.

41. The method of claim 17, further comprising administering an EZH2 inhibitor or degrader.

42. The method of claim 19, further comprising administering an EZH2 inhibitor or degrader.

43. The method of claim 27, further comprising administering an EZH2 inhibitor or degrader.

44. (canceled)

45. (canceled)

46. (canceled)
